(19) United States
(12) Patent Application Publication
(10) Pub. No.: US 2025/0312433 A1
(43) Pub. Date: Oct. 9, 2025

(54) Process for Producing of Vaccine Formulations With Preservatives

(71) Applicant: PFIZER INC., New York, NY (US)

(72) Inventors: Michael David BRUCHSALER, St. Charles, MO (US); Christopher John GLOVER, O'Fallon, MO (US); Alex Jacob LANGFORD, St. Louis, MO (US)

(21) Appl. No.: 18/863,176

(22) PCT Filed: May 8, 2023
(86) PCT No.: PCT/IB2023/054758

§ 371 Date: Nov. 5, 2024

Related U.S. Application Data
(60) Provisional application No. 63/340,620, filed on May 11, 2022.
(60) Provisional application No. 63/494,514, filed on Apr. 6, 2023.

(51) Int. Cl. A61K 39/09; A61K 39/00; A61K 39/385; A61K 47/02; A61K 47/10; A61K 47/12; A61K 47/26; A61K 47/64

(52) U.S. Cl. CPC A61K 39/092; A61K 39/385; A61K 47/02; A61K 47/10; A61K 47/12; A61K 47/26; A61K 47/646; A61K 2039/555; A61K 2039/6037; A61K 2039/70

(57) ABSTRACT

The present invention relates to a process for the production of a conjugate vaccine comprising a preservative. The invention relates in particular to a process for the production of a conjugate vaccine where the preservative is hydrophobic and viscous (such as 2-phenoxyethanol (2-PE)).

### FIELD OF THE INVENTION

The present invention relates to a process for the production of a conjugate vaccine comprising a preservative. The invention relates in particular to a process for the production of a conjugate vaccine where the preservative is hydrophobic and viscous (such as 2-phenoxyethanol (2-PE)).

### BACKGROUND OF THE INVENTION

Bacterial cell surface polysaccharides, particularly capsular polysaccharides, have become increasingly important as therapeutic agents. Typically, a cell surface polysaccharide is associated with inducing an immune response in vivo.

Although polysaccharides are immunogenic on their own, conjugation of polysaccharides to protein carriers (glycoconjugate) has been used to improve immunogenicity, particularly in infants and the elderly. Glycoconjugate vaccines are typically obtained by covalent linkage of poorly immunogenic sugar antigens to a protein carrier and play an important role in the prevention of many deadly infectious diseases. In the preparation of conjugate vaccines, selected bacterial strains are grown to supply polysaccharides needed to produce the vaccine. The cells are often grown in fermentors with lysis induced at the end of the fermentation. The lysate broth is then harvested for downstream purification and the recovery of the capsular polysaccharide which surrounds the bacterial cells. After conjugation with a carrier protein, the polysaccharide is included in the final vaccine product and confers immunity in the vaccine's target population to the bacteria.

Pneumococcal disease caused by Streptococcus pneumoniae is one of the more important bacterial pathogens across globe. Pneumococcal disease is a complex group of illnesses and includes invasive infections such as bacteremia/sepsis, meningitis, pneumonia and otitis media, which affects both children and adults. Prevnar 13 (also known as “Prevenar 13” and referred to herein as “Prev(e)nar 13”) is a formulation of polysaccharides from thirteen pneumococcal serotypes (1, 3, 4, 5, 6A, 6B, 7F, 9V, 14, 18C, 19A, 19F and 23F)) which are individually conjugated to CRM₁₉₇ (Cross Reactive Material from a mutant strain of Corynebacterim diphtheriae). See e.g., WO2006/110381; WO2008/079653; WO2008/079732; WO2008/143709 and WO 2011/151760.

The need for adding preservatives to vaccines can be reduced or obviated by making and using only single-dose vaccine formulations. However, use of single-dose preservative-free formulations raises the overall cost of vaccination and jeopardizes the effectiveness of immunization programs especially in developing countries. In addition, removing preservatives from multi-dose vials altogether is not viewed as a preferred option, especially in countries with limited cold storage and suboptimal standards of health care (Drain et al., Bull World Health Organ 81(10): 726-731 (2003). Thus, although multi-dose vials appear to be most appropriate for the production of less expensive vaccines, it is desirable to formulate multi-dose vaccines with at least one preservative to protect subjects from micro-organisms inadvertently introduced into the vaccine during multiple uses or after one or more non-sterile events.

WO 2011/151760 discloses a multivalent immunogenic composition comprising a plurality of capsular polysaccharides from Streptococcus pneumoniae serotypes and 2-phenoxyethanol (2-PE).

The inventors have found that formulating a conjugate vaccine with a preservative such as 2-PE poses significant challenges, especially at a large scale. In the prior art 2-PE has been added at final stage of vaccine formulation (to drug product see e.g. WO 00/62801, WO 00/56360 or Khandke L. et al Vaccine 29 (2011) 7144-7153). However, adding 2-PE at final stage requires sterile filtration of the 2-PE at a relatively high concentration. It has been found that sterile filtration of 2-PE at such high concentration poses challenges with filter wetting and pre-use filter integrity.

Accordingly, improved process for formulating a multivalent conjugate vaccine comprising a preservative, such as 2-PE are needed. In particular, processes which are less time consuming and with reduced number of steps are desirable.

### SUMMARY OF THE INVENTION

In an aspect, the invention provides a process for the production of a conjugate vaccine comprising a preservative, said process comprising the steps of:
(a) adding a preservative from a bulk solution to a solution comprising one or more conjugate(s), wherein said preservative is added without sterile filtration; and
(b) subsequently sterile filter the mixture comprising said preservative and said conjugate(s).

In an aspect, the process further comprises the step of (c) subsequently adding an adjuvant.

In an aspect, the preservative is hydrophobic and viscous at the concentration of the bulk solution.

Preferably, the bulk solution is neat 2-PE.

### DETAILED DESCRIPTION OF THE INVENTION

An approach used to add preservatives to complex vaccine formulations consists in adding the preservatives at the final stage of the formulation process. Adding the preservative as the final stage of the process minimizes the risk of potential effect of interaction of the preservative with other components of the vaccine (such as the antigens) and allows for flexibility of the process.

Preservatives are defined as compounds added to vaccines to kill or to prevent the growth of microorganisms, particularly bacteria and fungi. They are added to vaccine formulations to prevent microbial growth in the event that the vaccine is accidentally contaminated, as might occur with repeated puncture of multi-dose vials.

The present inventors have found that adding preservative, in particular hydrophobic and viscous preservatives as one of the last steps of the process can be challenging, especially at a large scale. It has been found that due to the hydrophobicity and high viscosity of some preservatives (such as 2-PE), it is challenging to filter said preservative through sterile filter at the required concentration, a step which is required if preservative is added at the end of the formulation process. It has been found that an ultra-high pressure would be required to completely and consistently wet out the sterile filter (i.e. a filter having a nominal pore size of about 0.2 μm) with the preservative at the required concentration, which could compromise the microbial retention property of the membrane. In case such high pressure is not used, wetting the filter with the preservative at the required concentration may become incomplete and inconsistent, which means that the wetting step may fail and requires special testing to ensure wetting is appropriate and potential waste of material. Such testing can therefore be time consuming and source of waste.

Accordingly, an improved process for formulating a multivalent conjugate vaccine comprising a preservative, such as 2-PE is needed. In particular, the process should be less time consuming (preparation of and with reduced number of steps are desirable).

It has been surprisingly found that adding the preservative earlier in the process has advantages. In such a process, since the preservative is added earlier (prior to final dilution), the preservative is present at a higher concentration than in the final drug product. It has been surprisingly found that adding the preservative earlier in the process, in particular before sterile filtration is required, is possible and allows avoiding the issues associated with sterile filtration of hydrophobic and viscous preservatives at high concentration as only diluted solution is required to be sterile filtered downstream. Such a process allows to add the preservative without sterile filtration. Sterile filtration of hydrophobic and viscous preservatives has been found problematic (in particular for 2-PE).

Therefore, the present invention relates to a process for the production of a conjugate vaccine comprising a preservative, said process comprising the steps of
(a) adding a preservative from a bulk solution to a solution comprising one or more conjugate(s), wherein said preservative is added without sterile filtration; and
(b) subsequently sterile filter the mixture comprising said preservative and said conjugate(s).

In an embodiment, the sterile filtration step (b) is further followed by the addition of an adjuvant. Therefore, in an embodiment, the invention relates to a process for the production of a conjugate vaccine comprising a preservative, said process comprising the steps of
(a) adding a preservative from a bulk solution to a solution comprising one or more conjugate(s), wherein said preservative is added without sterile filtration;
(b) subsequently sterile filter the mixture comprising said preservative and said conjugate(s) and;
(c) subsequently adding an adjuvant to said mixture.

Preferably, the preservative used in the present invention is hydrophobic and viscous at the concentration of the bulk solution. In an embodiment, the viscosity of the preservative at the concentration of the bulk solution is at least 10 centistokes at 25° C. In another embodiment, said viscosity is at least 15 centistokes at 25° C. In a preferred embodiment said viscosity is at least 20 centistokes at 25° C.

In an embodiment, the viscosity of the preservative used in the present invention is between about 10 to about 50 centistokes at 25° C. Preferably, the viscosity of the preservative used in the present invention is between about 15 to about 25 centistokes at 25° C.

In an embodiment, the preservative is 2-phenoxyethanol (2-PE), phenol, meta-cresol, methyl-paraben, propyl-paraben or thiomersal. In a preferred embodiment, the preservative is 2-phenoxyethanol (2-PE) or thiomersal.

In an embodiment, the preservative is added undiluted. Therefore, in an embodiment, the preservative is added undiluted (i.e. the bulk solution is the undiluted preservative). In an embodiment, the bulk solution is therefore undiluted pure preservative (where “pure” refers to pharmaceutical grade).

In a most preferred embodiment, the preservative used in the present invention is 2-phenoxyethanol (2-PE). 2-PE is a colorless oily liquid and can be added pure (undiluted at pharmaceutical grade). Therefore, in an embodiment, 2-PE is added undiluted (i.e. the bulk solution is neat 2-PE). In an embodiment, the bulk solution is therefore neat 2-PE.

In an embodiment, the bulk solution is neat 2-PE and the 2-PE is diluted by between about 10-fold and about 200-fold in the solution following step (a). In another embodiment, the bulk solution is neat 2-PE and the 2-PE is diluted by between about 10-fold and about 100-fold in the solution following step (a). In another embodiment, the bulk solution is neat 2-PE and the 2-PE is diluted by about 50-fold, about 60-fold, about 70-fold, or about 80-fold in the solution following step (a). In a preferred embodiment, the bulk solution is neat 2-PE and the 2-PE is diluted by between about 73-fold and 74-fold in the solution following step (a).

In an embodiment, the concentration of 2-PE in the solution following step (a) is between about 1 mg/ml and about 25 mg/ml. Preferably, the concentration of 2-PE in the solution following step (a) is between about 10 mg/ml and about 20 mg/ml. Even more preferably, the concentration of 2-PE in the solution following step (a) is between about 15 mg/ml and about 17 mg/ml.

In a particular embodiment, the concentration of 2-PE in the solution following step (a) is about 15 mg/ml.

In a particular embodiment, the concentration of 2-PE in the solution following step (a) is about 17 mg/ml.

In an embodiment, 2-PE is added at a rate of addition of between about 0.5 ml/min per L of solution comprising one or more conjugate(s) to about 5.0 ml/min per L of solution comprising one or more conjugate(s). In a preferred embodiment, 2-PE is added undiluted.

In an embodiment, 2-PE is added at a rate of addition of between about 1 ml/min per L of solution comprising one or more conjugate(s) to about 4.0 ml/min per L of solution comprising one or more conjugate(s). In a preferred embodiment, 2-PE is added undiluted (neat 2-PE).

In an embodiment, neat 2-PE is added using a pump, preferably a peristaltic pump. In an embodiment, neat 2-PE is added using a peristaltic pump using thermoplastic elastomer tubing. Preferably, neat 2-PE is added using a peristaltic pump using thermoplastic elastomer tubing.

In an embodiment, the final concentration of 2-PE in the vaccine is between about 5 mg/ml to about 15 mg/ml. Preferably, the final concentration of 2-PE in the vaccine is between about 7 mg/ml to about 12 mg/ml.

In an embodiment, the final concentration of 2-PE in the vaccine is about 10 mg/ml.

In a preferred embodiment, the final concentration of 2-PE in the vaccine is about 9 mg/ml.

In an embodiment, the volume of the solution following step (a) is between about 10 L and about 2,000 L. In another embodiment, the volume of the solution following step (a) is between about 100 L and about 1,000 L. In another embodiment, the volume of the solution following step (a) is between about 100 L and about 500 L. In another embodiment, the volume of the solution following step (a) is between about 150 L and about 200 L. In a preferred embodiment, the volume of the solution following step (a) is about 180 L.

Preferably, the preservative is added under continuous mixing. In a preferred embodiment, there is no delay between the addition of the preservative and the start of mixing.

In an embodiment, the preservative is added under continuous mixing where the angular velocity is between about 50 rpm to about 500 rpm. Preferably, the preservative is added under continuous mixing where the angular velocity is between about 100 rpm to about 400 rpm. More preferably, the preservative is added under continuous mixing where the angular velocity is between about 150 rpm to about 300 rpm.

Most preferably, the preservative is added under continuous mixing where the angular velocity is between about 150 rpm to about 200 rpm.

In an embodiment, the preservative is added under continuous mixing where the angular velocity is about 150 rpm.

In an embodiment, the preservative is added under continuous mixing where the angular velocity is about 200 rpm.

In an embodiment, following the addition of preservative and before sterile filtration, the solution is mixed for between about 15 minutes to about 5 hours. Preferably, following the addition of preservative and before sterile filtration, the solution is mixed for between about 30 minutes to about 3 hours. Even more preferably, following the addition of preservative and before sterile filtration, the solution is mixed for between about 1 hour to about 3 hours.

In an embodiment, following the addition of preservative, the solution is mixed about 2 hours.

In an embodiment, following the addition of preservative and before sterile filtration, the solution is mixed at an angular velocity between about 50 rpm and about 500 rpm. Preferably, between about 100 rpm and about 400 rpm. More preferably, between about 150 rpm and about 300 rpm.

### Solution Comprising One or More Conjugate(s) of Step (a)

The solution to which the preservative is added comprises one or more conjugate(s) and may comprise other components.

For the purposes of the invention the term ‘conjugate’ or ‘glycoconjugate’ indicates a bacterial capsular saccharide linked to a carrier protein. In one embodiment a capsular saccharide is linked directly to a carrier protein. In a second embodiment a capsular saccharide is linked to a protein through a spacer/linker.

In an embodiment, the solution to which the preservative is added is a mono-valent composition. In such an embodiment, the solution comprises only one conjugate. In such an embodiment, said conjugate maybe a conjugated Haemophilus influenzae type b capsular saccharide (Hib). In another embodiment, said conjugate is a conjugated N. meningitidis serogroup Y capsular saccharide (MenC). In yet another embodiment, said conjugate is a conjugated N. meningitidis serogroup Y capsular saccharide (MenY).

In an embodiment, the solution to which the preservative is added is a bi-valent, tri-valent, tetra-valent, pent-valent or hexa-valent composition.

In an embodiment, said tri-valent composition comprises three conjugated Streptococcus agalactiae capsular saccharides. Streptococcus agalactiae is also known as ‘group B Streptococcus’, or simply as ‘GBS’. In an embodiment, said three conjugates consists of conjugated GBS serotypes Ia, Ib and III capsular saccharides. In a preferred embodiment, said capsular saccharide are conjugated to CRM₁₉₇.

In an embodiment, said hexa-valent composition comprises six conjugated Streptococcus agalactiae capsular saccharides. In an embodiment, said six conjugates consists of conjugated GBS serotypes Ia, Ib, II, III, IV and V capsular saccharides. In a preferred embodiment, said capsular saccharides are conjugated to CRM₁₉₇.

In an embodiment, said tetra-valent composition comprises four conjugated N. meningitidis capsular saccharides. In an embodiment, said four conjugates consists of a conjugated N. meningitidis serogroup A capsular saccharide (MenA), a conjugated N. meningitidis serogroup W135 capsular saccharide (MenW135), a conjugated N. meningitidis serogroup Y capsular saccharide (MenY), and a conjugated N. meningitidis serogroup C capsular saccharide (MenC).

Preferably, the solution to which the preservative is added is a multi-valent composition comprising seven conjugates or more. Preferably, the solution is a 7 to 25 valent composition (i.e., it contains 7 to 25 conjugates). Even more preferably, the multi-valent solution is a 13 to 25 valent composition.

In an embodiment, the solution to which the preservative is added is a 15-valent composition.

In an embodiment, the solution to which the preservative is added is a 16-valent composition.

In an embodiment, the solution to which the preservative is added is a 20-valent composition.

In an embodiment, the solution to which the preservative is added is a 21-valent composition.

Preferably, the solution to which the preservative is added is a multi-valent pneumococcal conjugates composition.

In an embodiment, the solution to which the preservative is added is a pneumococcal conjugate composition comprising from 7 to 25 glycoconjugates from different serotypes of S. pneumoniae (7 to 25 pneumococcal conjugates).

In an embodiment, the solution to which the preservative is added is a pneumococcal conjugate composition comprising conjugates from 15, 16, 17, 18, 19, 20, 21, 22, 23, 24 or 25 different serotypes of S. pneumoniae.

In an embodiment, the solution to which the preservative is added is a pneumococcal conjugate composition comprising conjugates from 20 different serotypes of S. pneumoniae.

In an embodiment, the solution to which the preservative is added is a 7, 8, 9, 10, 11, 12, 13, 14, 15, 16, 17, 18, 19 or 20-valent pneumococcal conjugates composition.

In an embodiment, the solution to which the preservative is added is a 15-valent pneumococcal conjugates composition.

In an embodiment, the solution to which the preservative is added is a 20-valent pneumococcal conjugates composition.

In an embodiment, the solution to which the preservative is added is a 21-valent pneumococcal conjugates composition.

In an embodiment, the solution to which the preservative is added is a 22-valent pneumococcal conjugates composition.

In an embodiment, the solution to which the preservative is added is a 21, 22, 23, 24 or 25-valent pneumococcal conjugates composition.

In an embodiment, the solution to which the preservative is added comprises glycoconjugates from S. pneumoniae serotypes 4, 6B, 9V, 14, 18C, 19F and 23F. In an embodiment, the solution to which the preservative is added comprises glycoconjugates from S. pneumoniae serotypes 1, 4, 5, 6B, 7F, 9V, 14, 18C, 19F and 23F.

In an embodiment, the solution to which the preservative is added comprises glycoconjugates from S. pneumoniae serotypes 1, 3, 4, 5, 6B, 7F, 9V, 14, 18C, 19F and 23F.

In an embodiment, the solution to which the preservative is added comprises glycoconjugates from S. pneumoniae serotypes 1, 3, 4, 5, 6A, 6B, 7F, 9V, 14, 18C, 19A, 19F and 23F.

In an embodiment, the solution to which the preservative is added comprises glycoconjugates from S. pneumoniae serotypes 1, 3, 4, 5, 6A, 6B, 7F, 9V, 14, 18C, 19A, 19F, 22F, 23F and 33F.

In an embodiment, the solution to which the preservative is added comprises glycoconjugates from S. pneumoniae serotypes 1, 3, 4, 5, 6A, 6B, 7F, 8, 9V, 10A, 11A, 12F, 14, 15B, 18C, 19A, 19F, 22F, 23F and 33F.

In an embodiment, the solution to which the preservative is added comprises glycoconjugates from S. pneumoniae serotypes 1, 2, 3, 4, 5, 6A, 6B, 7F, 8, 9V, 10A, 11A, 12F, 14, 15B, 18C, 19A, 19F, 22F, 23F and 33F.

In an embodiment, the solution to which the preservative is added comprises glycoconjugates from S. pneumoniae serotypes 1, 3, 4, 5, 6A, 6B, 7F, 8, 9N, 9V, 10A, 11A, 12F, 14, 15B, 18C, 19A, 19F, 22F, 23F and 33F.

In an embodiment, the solution to which the preservative is added is a 10-valent pneumococcal conjugate composition wherein said 10 glycoconjugates are from S. pneumoniae serotypes 1, 4, 5, 6B, 7F, 9V, 14, 18C, 19F and 23F.

In an embodiment, the solution to which the preservative is added is a 13-valent pneumococcal conjugate composition wherein said 13 glycoconjugates are from S. pneumoniae serotypes 1, 3, 4, 5, 6A, 6B, 7F, 9V, 14, 18C, 19A, 19F and 23F.

In an embodiment, the solution to which the preservative is added is a 15-valent pneumococcal conjugate composition wherein said 15 glycoconjugates are from S. pneumoniae serotypes 1, 3, 4, 5, 6A, 6B, 7F, 9V, 14, 18C, 19A, 19F, 22F, 23F and 33F.

In an embodiment, the solution to which the preservative is added is a 20-valent pneumococcal conjugate composition wherein said 20 glycoconjugates are from S. pneumoniae serotypes 1, 3, 4, 5, 6A, 6B, 7F, 8, 9V, 10A, 11A, 12F, 14, 15B, 18C, 19A, 19F, 22F, 23F and 33F.

In an embodiment, the solution to which the preservative is added is a 21-valent pneumococcal conjugate composition wherein said 21 glycoconjugates are from S. pneumoniae serotypes 1, 2, 3, 4, 5, 6A, 6B, 7F, 8, 9V, 10A, 11A, 12F, 14, 15B, 18C, 19A, 19F, 22F, 23F and 33F.

In an embodiment, the solution to which the preservative is added is a 21-valent pneumococcal conjugate composition wherein said 21 glycoconjugates are from S. pneumoniae serotypes 1, 3, 4, 5, 6A, 6B, 7F, 8, 9N, 9V, 10A, 11A, 12F, 14, 15B, 18C, 19A, 19F, 22F, 23F and 33F.

In an embodiment, the solution to which the preservative is added is a 22-valent pneumococcal conjugate composition wherein said 22 glycoconjugates are from S. pneumoniae serotypes 1, 2, 3, 4, 5, 6A, 6B, 7F, 8, 9N, 9V, 10A, 11A, 12F, 14, 15B, 18C, 19A, 19F, 22F, 23F and 33F.

In an embodiment, the solution to which the preservative is added is a 21-valent pneumococcal conjugate composition wherein said 21 glycoconjugates are from S. pneumoniae serotypes 1, 3, 4, 5, 6A, 6B, 7F, 8, 9V, 10A, 11A, 12F, 14, 15A, 15B, 18C, 19A, 19F, 22F, 23F and 33F.

In an embodiment, the solution to which the preservative is added is a 21-valent pneumococcal conjugate composition wherein said 21 glycoconjugates are from S. pneumoniae serotypes 1, 3, 4, 5, 6A, 6B, 7F, 8, 9V, 10A, 11A, 12F, 14, 15B, 18C, 19A, 19F, 22F, 23A, 23F and 33F.

In an embodiment, the solution to which the preservative is added is a 21-valent pneumococcal conjugate composition wherein said 21 glycoconjugates are from S. pneumoniae serotypes 1, 3, 4, 5, 6A, 6B, 7F, 8, 9V, 10A, 11A, 12F, 14, 15B, 18C, 19A, 19F, 22F, 23B, 23F and 33F.

In an embodiment, the solution to which the preservative is added is a 21-valent pneumococcal conjugate composition wherein said 21 glycoconjugates are from S. pneumoniae serotypes 1, 3, 4, 5, 6A, 6B, 7F, 8, 9V, 10A, 11A, 12F, 14, 15B, 18C, 19A, 19F, 22F, 23F, 24F and 33F.

In an embodiment, the solution to which the preservative is added is a 21-valent pneumococcal conjugate composition wherein said 21 glycoconjugates are from S. pneumoniae serotypes 1, 3, 4, 5, 6A, 6B, 7F, 8, 9V, 10A, 11A, 12F, 14, 15B, 18C, 19A, 19F, 22F, 23F, 33F and 35B.

In a preferred embodiment, the solution to which the preservative is added is a 20-valent pneumococcal conjugate composition wherein said 20 glycoconjugates are from S. pneumoniae serotypes 1, 3, 4, 5, 6A, 6B, 7F, 8, 9V, 10A, 11A, 12F, 14, 15B, 18C, 19A, 19F, 22F, 23F and 33F.

A component of the glycoconjugate is a carrier protein to which the saccharide is conjugated. The terms “protein carrier” or “carrier protein” or “carrier” may be used interchangeably herein. Carrier proteins should be amenable to standard conjugation procedures. In an embodiment, the carrier protein of the glycoconjugate of the invention is DT (Diphtheria Toxoid), TT (Tetanus Toxoid), fragment C of TT, CRM₁₉₇ detoxified pneumolysin, PhtA, PhtB, PhtD, PhtE, PhtDE fusion, PhtBE fusion, OMPC, Por), PD (Haemophilus influenzae protein D) or a C5a peptidase from Streptococcus

(SCP). In an embodiment, the carrier protein of the glycoconjugate of the invention is DT (Diphtheria toxoid). In another embodiment, the carrier protein of the glycoconjugate of the invention is TT (tetanus toxoid). In another embodiment, the carrier protein of the glycoconjugate of the invention is PD (H. influenzae protein D; see, e.g., EP0594610 B).

In a preferred embodiment, the carrier protein of the glycoconjugate of the invention is TT, CRM₁₉₇ or a C5a peptidase from Streptococcus (SCP). In an even more preferred embodiment, the carrier protein of the glycoconjugate of the invention is CRM197 or a C5a peptidase from Streptococcus (SCP).

In a very preferred embodiment, the carrier protein of the glycoconjugate of the invention is CRM₁₉₇. In an embodiment of any of the above compositions, the capsular polysaccharides are all individually conjugated to CRM₁₉₇.

In an embodiment, the capsular polysaccharides from S. pneumoniae serotypes 1, 4, 5, 6B, 7F, 9V, 14 and/or 23F of any of the above compositions are individually conjugated to PD, the capsular polysaccharide from S. pneumoniae serotype 18C is conjugated to TT and the capsular polysaccharide from S. pneumoniae serotype 19F is conjugated to DT.

In an embodiment of any of the above compositions, at least one capsular polysaccharide is conjugated to TT and the other capsular polysaccharides are all conjugated to CRM₁₉₇.

In an embodiment of any of the above compositions, one capsular polysaccharide is conjugated to TT and the other capsular polysaccharides are all conjugated to CRM₁₉₇.

In an embodiment of any of the above compositions, at least two capsular polysaccharides are conjugated to TT and the other capsular polysaccharides are all conjugated to CRM₁₉₇.

In an embodiment of any of the above compositions, two capsular polysaccharides are conjugated to TT and the other capsular polysaccharides are all conjugated to CRM₁₉₇.

In an embodiment of any of the above compositions, at least three capsular polysaccharides are conjugated to TT and the other capsular polysaccharides are all conjugated to CRM₁₉₇.

In an embodiment of any of the above compositions, three capsular polysaccharides are conjugated to TT and the other capsular polysaccharides are all conjugated to CRM₁₉₇.

In an embodiment of any of the above compositions, four capsular polysaccharides are conjugated to TT and the other capsular polysaccharides are all conjugated to CRM₁₉₇.

In an embodiment the solution to which the preservative is added is a 21-valent pneumococcal conjugate composition wherein said 21 glycoconjugates are from S. pneumoniae serotypes 1, 3, 4, 5, 6A, 6B, 7F, 8, 9N, 9V, 10A, 11A, 12F, 14, 15B, 18C, 19A, 19F, 22F, 23F and 33F, wherein four capsular polysaccharides are conjugated to TT wherein the four capsular polysaccharides that are conjugated to TT are serotypes 15B, 22F and the other two serotypes are selected from the group consisting of serotypes 1, 3 and 5 and the other glycoconjugates are all conjugated to CRM₁₉₇.

In an embodiment the solution to which the preservative is added is a 21-valent pneumococcal conjugate composition wherein said 21 glycoconjugates are from S. pneumoniae serotypes 1, 3, 4, 5, 6A, 6B, 7F, 8, 9N, 9V, 10A, 11A, 12F, 14, 15B, 18C, 19A, 19F, 22F, 23F and 33F, wherein four capsular polysaccharides are conjugated to TT wherein the four capsular polysaccharides that are conjugated to TT are serotypes 1, 5, 15B and 22F and the other glycoconjugates are all conjugated to CRM₁₉₇.

In an embodiment the solution to which the preservative is added is a 21-valent pneumococcal conjugate composition wherein said 21 glycoconjugates are from S. pneumoniae serotypes 1, 3, 4, 5, 6A, 6B, 7F, 8, 9N, 9V, 10A, 11A, 12F, 14, 15B, 18C, 19A, 19F, 22F, 23F and 33F, wherein four capsular polysaccharides are conjugated to TT wherein the four capsular polysaccharides that are conjugated to TT are serotypes 1, 3, 15B and 22F and the other glycoconjugates are all conjugated to CRM₁₉₇.

In an embodiment the solution to which the preservative is added is a 21-valent pneumococcal conjugate composition wherein said 21 glycoconjugates are from S. pneumoniae serotypes 1, 3, 4, 5, 6A, 6B, 7F, 8, 9N, 9V, 10A, 11A, 12F, 14, 15B, 18C, 19A, 19F, 22F, 23F and 33F, wherein four capsular polysaccharides are conjugated to TT wherein the four capsular polysaccharides that are conjugated to TT are serotypes 3, 5, 15B and 22F and the other glycoconjugates are all conjugated to CRM₁₉₇.

In an embodiment the solution to which the preservative is added is a 21-valent pneumococcal conjugate composition wherein said 21 glycoconjugates are from S. pneumoniae serotypes 1, 3, 4, 5, 6A, 6B, 7F, 8, 9N, 9V, 10A, 11A, 12F, 14, 15B, 18C, 19A, 19F, 22F, 23F and 33F, wherein two glycoconjugates selected from serotypes 1, 3 and 5 are conjugated to TT and the other glycoconjugates are all conjugated to CRM₁₉₇.

In an embodiment the solution to which the preservative is added is a 21-valent pneumococcal conjugate composition wherein said 21 glycoconjugates are from S. pneumoniae serotypes 1, 3, 4, 5, 6A, 6B, 7F, 8, 9N, 9V, 10A, 11A, 12F, 14, 15B, 18C, 19A, 19F, 22F, 23F and 33F, wherein glycoconjugates from serotypes 1 and 3 are conjugated to TT and the other glycoconjugates are all conjugated to CRM₁₉₇.

In an embodiment the solution to which the preservative is added is a 21-valent pneumococcal conjugate composition wherein said 21 glycoconjugates are from S. pneumoniae serotypes 1, 3, 4, 5, 6A, 6B, 7F, 8, 9N, 9V, 10A, 11A, 12F, 14, 15B, 18C, 19A, 19F, 22F, 23F and 33F, wherein glycoconjugates from serotypes 1 and 5 are conjugated to TT and the other glycoconjugates are all conjugated to CRM₁₉₇.

In an embodiment the solution to which the preservative is added is a 21-valent pneumococcal conjugate composition wherein said 21 glycoconjugates are from S. pneumoniae serotypes 1, 3, 4, 5, 6A, 6B, 7F, 8, 9N, 9V, 10A, 11A, 12F, 14, 15B, 18C, 19A, 19F, 22F, 23F and 33F, wherein glycoconjugates from serotypes 3 and 5 are conjugated to TT and the other glycoconjugates are all conjugated to CRM₁₉₇.

In an embodiment the solution to which the preservative is added is a 15-valent pneumococcal conjugate composition wherein said 15 glycoconjugates are from S. pneumoniae serotypes 1, 3, 4, 5, 6A, 6B, 7F, 9V, 14, 18C, 19A, 19F, 22F, 23F and 33F, wherein all the glycoconjugates are conjugated to CRM₁₉₇.

In a most preferred embodiment, the solution to which the preservative is added is a 20-valent pneumococcal conjugate composition wherein said 20 glycoconjugates are from S. pneumoniae serotypes 1, 3, 4, 5, 6A, 6B, 7F, 8, 9V, 10A, 11A, 12F, 14, 15B, 18C, 19A, 19F, 22F, 23F and 33F, wherein all the glycoconjugates are conjugated to CRM₁₉₇.

In an embodiment, the solution to which the preservative is added comprises any of the combination of glycoconjugate(s) disclosed herein and a pharmaceutically acceptable excipient, carrier, or diluent.

In addition to the conjugate(s) the solution to which the preservative is added may comprise one or more of a buffer, a salt, a divalent cation, a non-ionic detergent, a cryoprotectant such as a sugar, and an anti-oxidant such as a free radical scavenger or chelating agent, or any multiple combinations thereof.

In an embodiment, the solution to which the preservative is added comprises a buffer. In an embodiment, said buffer has a pKa of about 3.5 to about 7.5. In some embodiments, the buffer is phosphate, succinate, histidine or citrate. In some embodiments, the buffer is succinate. In a preferred embodiment, the buffer is histidine.

In certain embodiments, the buffer is histidine at a concentration of 10 mM to 30 mM. In certain embodiments, the buffer is histidine at a concentration of 15 mM to 25 mM. In one particular embodiment, the concentration of the histidine buffer is about 22 mM.

In certain embodiments, the buffer is succinate at a concentration of 1 mM to 10 mM. In one particular embodiment, the concentration of the succinate buffer is about 5.5 mM.

In an embodiment, the solution to which the preservative is added comprises a salt. In some embodiments, the salt is magnesium chloride, potassium chloride, sodium chloride or a combination thereof. In one particular embodiment, the salt is sodium chloride. In one particular embodiment, the solution to which the preservative is added comprises sodium chloride at about 150 mM.

In an embodiment, the solution to which the preservative is added comprises a surfactant. In an embodiment, the surfactant is polysorbate 20 (TWEEN™20), polysorbate 40 (TWEEN™40), polysorbate 60 (TWEEN™60), polysorbate 65 (TWEEN™65), polysorbate 80 (TWEEN™80), polysorbate 85 (TWEEN™85), TRITON™ N-101, TRITON™ X-100, oxtoxynol 40, nonoxynol-9, triethanolamine, triethanolamine polypeptide oleate, polyoxyethylene-660 hydroxystearate (PEG-15, Solutol H 15), polyoxyethylene-35-ricinoleate (CREMOPHOR® EL), soy lecithin or a poloxamer.

In a preferred embodiment, the surfactant is polysorbate 80 or polysorbate 20.

In one particular embodiment, the surfactant is polysorbate 20. Preferably, the concentration of polysorbate 20 in the solution is between 0.01% to 1% polysorbate 20 weight to weight (w/w). More preferably, the concentration of polysorbate 20 in the solution is between 0.1% to 0.5% polysorbate 20 weight to weight (w/w).

In other embodiments, the concentration of polysorbate 20 in the solution is about 0.1%, about 0.2%, about 0.3%, about 0.4%, about 0.5%, or about 0.6% polysorbate 20 (w/w). In a most preferred embodiment, the concentration of the polysorbate 20 in the solution is about 0.33% polysorbate 20 (w/w).

In a preferred embodiment, the surfactant is polysorbate 80. Preferably, the concentration of polysorbate 80 in the solution is between 0.001% to 1% polysorbate 80 weight to weight (w/w). More preferably, the concentration of polysorbate 80 in the solution is between 0.01% to 0.5% polysorbate 80 weight to weight (w/w).

In other embodiments, the concentration of polysorbate 80 in the solution is about 0.01%, about 0.02%, about 0.03%, about 0.04%, about 0.05%, or about 0.06% polysorbate 80 (w/w). In a most preferred embodiment, the concentration of the polysorbate 80 in the solution is about 0.033% polysorbate 80 (w/w).

In certain embodiments, the solution to which the preservative is added has a pH of between 5.5 to 7.5, more preferably a pH of 5.6 to 7.0, even more preferably a pH of 5.8 to 6.0. In a preferred embodiment, the solution to which the preservative is added a pH of about 5.8.

The concentration of a particular glycoconjugate can be calculated based on the amount of polysaccharide for that conjugate. The saccharide concentration can be determined by the uronic acid assay.

In an embodiment, the solution to which the preservative is added comprises between about 2 μg/ml to about 20 μg/ml of polysaccharide for glycoconjugates from S. pneumoniae serotype 1, 2, 3, 4, 5, 6A, 7F, 8, 9N, 9V, 10A, 11A, 12F, 14, 15A, 15B, 18C, 19A, 19F, 22F, 23A, 23B, 23F, 24F, 33F and/or 35B. Preferably, between about 5 μg/ml to about 10 μg/ml.

In an embodiment, the solution to which the preservative is added comprises about 7.5 μg/ml of polysaccharide for glycoconjugates from S. pneumoniae serotype 1, 2, 3, 4, 5, 6A, 7F, 8, 9N, 9V, 10A, 11A, 12F, 14, 15A, 15B, 18C, 19A, 19F, 22F, 23A, 23B, 23F, 24F, 33F and/or 35B.

In an embodiment, the solution to which the preservative is added comprises between about 4 μg/ml to about 40 μg/ml of polysaccharide for glycoconjugate from S. pneumoniae serotype 6B. Preferably, between about 10 μg/ml to about 20 g/ml.

In an embodiment, the solution to which the preservative is added comprises about 15 μg/ml of polysaccharide for glycoconjugate from S. pneumoniae serotype 6B.

In an embodiment, the solution to which the preservative is added is a 20-valent pneumococcal conjugate composition wherein said 20 glycoconjugates are from S. pneumoniae serotypes 1, 3, 4, 5, 6A, 6B, 7F, 8, 9V, 10A, 11A, 12F, 14, 15B, 18C, 19A, 19F, 22F, 23F and 33F, wherein the glycoconjugates are all conjugated to CRM₁₉₇, which comprises between about 5 μg/ml to about 10 μg/ml of each of capsular polysaccharides serotypes 1, 3, 4, 5, 6A, 7F, 8, 9V, 10A, 11A, 12F, 14, 15B, 18C, 19A, 19F, 22F, 23F, and 33F and between about 10 μg/ml to about 20 μg/ml of the capsular polysaccharide of serotype 6B, between about 0.01% to about 0.05% (w/w) polysorbate 80, about 150 mM sodium chloride and between about 1 mM to about 10 mM succinate buffer pH 5.8.

In an embodiment the solution to which the preservative is added is a 15-valent pneumococcal conjugate composition wherein said 15 glycoconjugates are from S. pneumoniae serotypes 1, 3, 4, 5, 6A, 6B, 7F, 9V, 14, 18C, 19A, 19F, 22F, 23F and 33F, wherein the glycoconjugates are all conjugated to CRM₁₉₇, which comprises between about 5 μg/ml to about 10 μg/ml of each of capsular polysaccharides serotypes 1, 3, 4, 5, 6A, 7F, 9V, 14, 18C, 19A, 19F, 22F, 23F, and 33F and between about 10 μg/ml to about 20 μg/ml of the capsular polysaccharide of serotype 6B, about 150 mM sodium chloride, between about 0.1% to about 0.5% (w/w) polysorbate 20 and between about 15 mM to about 25 mM L-Histidine buffer pH 5.8.

In a preferred embodiment the solution to which the preservative is added is a 20-valent pneumococcal conjugate composition wherein said 20 glycoconjugates are from S. pneumoniae serotypes 1, 3, 4, 5, 6A, 6B, 7F, 8, 9V, 10A, 11A, 12F, 14, 15B, 18C, 19A, 19F, 22F, 23F and 33F, wherein the glycoconjugates are all conjugated to CRM₁₉₇, which comprises about 7.5 μg/ml of each of capsular polysaccharides serotypes 1, 3, 4, 5, 6A, 7F, 8, 9V, 10A, 11A, 12F, 14, 15B, 18C, 19A, 19F, 22F, 23F, and 33F and about 7.5 μg/ml of the capsular polysaccharide of serotype 6B, about 0.033% (w/w) polysorbate 80, about 150 mM sodium chloride and about 5.5 mM succinate buffer pH 5.8.

### Sterile Filtration

Following step (a), the mixture comprising the preservative and the conjugate(s) is sterilely filtered.

In an embodiment, the filter has a nominal retention range of between about 0.05-0.2 μm or about 0.1-0.2 μm. Preferably, about 0.15-0.2 μm.

In an embodiment, the filter has a nominal retention range of about 0.1, about 0.15 or about 0.2 μm.

In a preferred embodiment, the filter has a nominal retention range of about 0.2 μm.

In a preferred embodiment, the filter comprises a prefilter. In some embodiments, the prefilter has a nominal retention range of between about 0.1 μm and about 1 μm. In a preferred embodiment, the prefilter has a nominal retention range of about 0.5 μm. In another preferred embodiment, the prefilter has a nominal retention range of about 0.5 μm and the filter has a nominal retention range of about 0.2 μm.

In an embodiment, the filter has a filter capacity of about 25-1000 L/m2, 50-1000 L/m2, 75-1000 L/m2, 100-1000 L/m2, 150-1000 L/m2, 200-1000 L/m2, 250-1000 L/m2, 300-1000 L/m2, 350-1000 L/m2, 400-1000 L/m2, 500-1000 L/m2 or 750-1000 L/m2.

In an embodiment, the filter has a filter capacity of 200-1000 L/m2, 250-1000 L/m2 or 300-1000 L/m2.

In a preferred embodiment, the filter has a filter capacity of about 300-1000 L/m2.

In an embodiment, the filter comprises polysulfone. In one embodiment, the filter comprises polyphenylene sulfone. In a preferred embodiment, the filter comprises polyethersulfone (PES). In an embodiment, the filter is a disc filter. In a preferred embodiment, the filter is a capsule filter. In a further preferred embodiment, the filter is a capsule filter that comprises PES.

### Conjugate Vaccine of the Invention

The conjugate vaccine obtained by the process of the invention comprises one or more conjugate and a preservative. Preferably, said preservative is 2-PE.

For the purposes of the invention the term ‘conjugate’ indicates a capsular saccharide linked to a carrier protein. In one embodiment a capsular saccharide is linked directly to a carrier protein. In a second embodiment a capsular saccharide is linked to a protein through a spacer/linker.

In an embodiment, the conjugate vaccine of the invention is a mono-valent conjugate vaccine. In such an embodiment, the conjugate vaccine comprises only one conjugate. In such an embodiment, said conjugate maybe is a conjugated Haemophilus influenzae type b capsular saccharide (Hib). In another embodiment, said conjugate is a conjugated N. meningitidis serogroup Y capsular saccharide (MenC). In yet another embodiment, said conjugate is a conjugated N. meningitidis serogroup Y capsular saccharide (MenY).

In another embodiment, the conjugate vaccine of the invention is a bi-valent, tri-valent, tetra-valent, pent-valent or hexa-valent conjugate vaccine.

In an embodiment, said tri-valent conjugate vaccine comprises three conjugated Streptococcus agalactiae capsular saccharides. Streptococcus agalactiae is also known as ‘group B Streptococcus’, or simply as ‘GBS’. In an embodiment, said three conjugates consists of conjugated GBS serotypes Ia, Ib and III capsular saccharides. In a preferred embodiment, said capsular saccharide are conjugated to CRM₁₉₇.

In an embodiment, said hexa-valent conjugate vaccine comprises six conjugated Streptococcus agalactiae capsular saccharides. In an embodiment, said six conjugates consists of conjugated GBS serotypes Ia, Ib, II, III, IV and V capsular saccharides. In a preferred embodiment, said capsular saccharides are conjugated to CRM₁₉₇.

In an embodiment, said tetra-valent conjugate vaccine comprises four conjugated N. meningitidis capsular saccharides. In an embodiment, said four conjugates consists of a conjugated N. meningitidis serogroup A capsular saccharide (MenA), a conjugated N. meningitidis serogroup W135 capsular saccharide (MenW135), a conjugated N. meningitidis serogroup Y capsular saccharide (MenY), and a conjugated N. meningitidis serogroup C capsular saccharide (MenC).

In a preferred embodiment, the conjugate vaccine of the invention is a multi-valent conjugate vaccine comprising seven conjugates or more. Preferably, the multi-valent conjugate vaccine of the invention is a 7 to 25 valent conjugate vaccine (i.e. it contains seven to 25 conjugates). Even more preferably, the multi-valent conjugate vaccine of the invention is a 13 to 25 valent conjugate vaccine

In an embodiment, the multi-valent conjugate vaccine of the invention is a 15-valent conjugate vaccine.

In an embodiment, the multi-valent conjugate vaccine of the invention is a 16-valent conjugate vaccine.

In an embodiment, the multi-valent conjugate vaccine of the invention is a 20-valent conjugate vaccine.

In an embodiment, the multi-valent conjugate vaccine of the invention is a 21-valent conjugate vaccine.

Preferably, the multi-valent conjugate vaccine of the invention is a pneumococcal conjugate vaccine.

In an embodiment, the multi-valent conjugate vaccine of the invention is a pneumococcal conjugate vaccine comprising from 7 to 25 glycoconjugates from different serotypes of S. pneumoniae (7 to 25 pneumococcal conjugates).

In an embodiment, the multi-valent conjugate vaccine of the invention is a pneumococcal conjugate vaccine comprising glycoconjugates from 15, 16, 17, 18, 19, 20, 21, 22, 23, 24 or 25 different serotypes of S. pneumoniae.
In an embodiment, the multi-valent conjugate vaccine of the invention is a pneumococcal conjugate vaccine comprising glycoconjugates from 20 different serotypes of S. pneumoniae.

In an embodiment, the pneumococcal conjugate vaccine of the invention is a 7, 8, 9, 10, 11, 12, 13, 14, 15, 16, 17, 18, 19 or 20-valent pneumococcal conjugate vaccine.

In an embodiment, the pneumococcal conjugate vaccine of the invention is a 15-valent pneumococcal conjugate vaccine.

In a preferred embodiment, the pneumococcal conjugate vaccine of the invention is a 20-valent pneumococcal conjugate vaccine.

In an embodiment, the pneumococcal conjugate vaccine of the invention is a 21, 22, 23, 24 or 25-valent pneumococcal conjugate vaccine.

In an embodiment, the pneumococcal conjugate vaccine of the invention is a 21-valent pneumococcal conjugate vaccine.

In an embodiment, the pneumococcal conjugate vaccine of the invention is a 22-valent pneumococcal conjugate vaccine.

In an embodiment, the pneumococcal conjugate vaccine of the invention comprises glycoconjugates from S. pneumoniae serotypes 4, 6B, 9V, 14, 18C, 19F and 23F.

In an embodiment, the pneumococcal conjugate vaccine of the invention comprises glycoconjugates from S. pneumoniae serotypes 1, 4, 5, 6B, 7F, 9V, 14, 18C, 19F and 23F.

In an embodiment, the pneumococcal conjugate vaccine of the invention comprises glycoconjugates from S. pneumoniae serotypes 1, 3, 4, 5, 6B, 7F, 9V, 14, 18C, 19F and 23F.

In an embodiment, the pneumococcal conjugate vaccine of the invention comprises glycoconjugates from S. pneumoniae serotypes 1, 3, 4, 5, 6A, 6B, 7F, 9V, 14, 18C, 19A, 19F and 23F.

In an embodiment, the pneumococcal conjugate vaccine of the invention comprises glycoconjugates from S. pneumoniae serotypes 1, 3, 4, 5, 6A, 6B, 7F, 9V, 14, 18C, 19A, 19F, 22F, 23F and 33F.

In an embodiment, the pneumococcal conjugate vaccine of the invention comprises glycoconjugates from S. pneumoniae serotypes 1, 3, 4, 5, 6A, 6B, 7F, 8, 9V, 10A, 11A, 12F, 14, 15B, 18C, 19A, 19F, 22F, 23F and 33F.

In an embodiment, the pneumococcal conjugate vaccine of the invention comprises glycoconjugates from S. pneumoniae serotypes 1, 2, 3, 4, 5, 6A, 6B, 7F, 8, 9V, 10A, 11A, 12F, 14, 15B, 18C, 19A, 19F, 22F, 23F and 33F.

In an embodiment, the pneumococcal conjugate vaccine of the invention comprises glycoconjugates from S. pneumoniae serotypes 1, 3, 4, 5, 6A, 6B, 7F, 8, 9N, 9V, 10A, 11A, 12F, 14, 15B, 18C, 19A, 19F, 22F, 23F and 33F.

In an embodiment the pneumococcal conjugate vaccine of the invention is a 10-valent pneumococcal conjugate wherein said 10 glycoconjugates are from S. pneumoniae serotypes 1, 4, 5, 6B, 7F, 9V, 14, 18C, 19F and 23F.

In an embodiment the pneumococcal conjugate vaccine of the invention is a 13-valent pneumococcal conjugate wherein said 13 glycoconjugates are from S. pneumoniae serotypes 1, 3, 4, 5, 6A, 6B, 7F, 9V, 14, 18C, 19A, 19F and 23F. In an embodiment the pneumococcal conjugate vaccine of the invention is a 15-valent pneumococcal conjugate wherein said 15 glycoconjugates are from S. pneumoniae serotypes 1, 3, 4, 5, 6A, 6B, 7F, 9V, 14, 18C, 19A, 19F, 22F, 23F and 33F.

In an embodiment the pneumococcal conjugate vaccine of the invention is a 20-valent pneumococcal conjugate wherein said 20 glycoconjugates are from S. pneumoniae serotypes 1, 3, 4, 5, 6A, 6B, 7F, 8, 9V, 10A, 11A, 12F, 14, 15B, 18C, 19A, 19F, 22F, 23F and 33F.

In an embodiment the pneumococcal conjugate vaccine of the invention is a 21-valent pneumococcal conjugate wherein said 21 glycoconjugates are from S. pneumoniae serotypes 1, 2, 3, 4, 5, 6A, 6B, 7F, 8, 9V, 10A, 11A, 12F, 14, 15B, 18C, 19A, 19F, 22F, 23F and 33F.

In an embodiment the pneumococcal conjugate vaccine of the invention is a 21-valent pneumococcal conjugate wherein said 21 glycoconjugates are from S. pneumoniae serotypes 1, 3, 4, 5, 6A, 6B, 7F, 8, 9N, 9V, 10A, 11A, 12F, 14, 15B, 18C, 19A, 19F, 22F, 23F and 33F.

In an embodiment the pneumococcal conjugate vaccine of the invention is a 22-valent pneumococcal conjugate wherein said 22 glycoconjugates are from S. pneumoniae serotypes 1, 2, 3, 4, 5, 6A, 6B, 7F, 8, 9N, 9V, 10A, 11A, 12F, 14, 15B, 18C, 19A, 19F, 22F, 23F and 33F.

In an embodiment the pneumococcal conjugate vaccine of the invention is a 21-valent pneumococcal conjugate wherein said 21 glycoconjugates are from S. pneumoniae serotypes 1, 3, 4, 5, 6A, 6B, 7F, 8, 9V, 10A, 11A, 12F, 14, 15A, 15B, 18C, 19A, 19F, 22F, 23F and 33F.

In an embodiment the pneumococcal conjugate vaccine of the invention is a 21-valent pneumococcal conjugate wherein said 21 glycoconjugates are from S. pneumoniae serotypes 1, 3, 4, 5, 6A, 6B, 7F, 8, 9V, 10A, 11A, 12F, 14, 15B, 18C, 19A, 19F, 22F, 23A, 23F and 33F.

In an embodiment the pneumococcal conjugate vaccine of the invention is a 21-valent pneumococcal conjugate wherein said 21 glycoconjugates are from S. pneumoniae serotypes 1, 3, 4, 5, 6A, 6B, 7F, 8, 9V, 10A, 11A, 12F, 14, 15B, 18C, 19A, 19F, 22F, 23B, 23F and 33F.

In an embodiment the pneumococcal conjugate vaccine of the invention is a 21-valent pneumococcal conjugate wherein said 21 glycoconjugates are from S. pneumoniae serotypes 1, 3, 4, 5, 6A, 6B, 7F, 8, 9V, 10A, 11A, 12F, 14, 15B, 18C, 19A, 19F, 22F, 23F 24F and 33F.

In an embodiment the pneumococcal conjugate vaccine of the invention is a 21-valent pneumococcal conjugate wherein said 21 glycoconjugates are from S. pneumoniae serotypes 1, 3, 4, 5, 6A, 6B, 7F, 8, 9V, 10A, 11A, 12F, 14, 15B, 18C, 19A, 19F, 22F, 23F, 33F and 35B.

A component of the glycoconjugate is a carrier protein to which the saccharide is conjugated. The terms “protein carrier” or “carrier protein” or “carrier” may be used interchangeably herein. Carrier proteins should be amenable to standard conjugation procedures.

In an embodiment, the carrier protein of the glycoconjugate of the invention is selected in the group consisting of: DT (Diphtheria Toxoid), TT (Tetanus Toxoid), fragment C of TT, CRM₁₉₇ (a nontoxic but antigenically identical variant of diphtheria toxin), other DT mutants (such as CRM176, CRM228, CRM45 (Uchida et al. (1973) J. Biol. Chem. 218:3838-3844), CRM9, CRM102, CRM103 or CRM107, pneumococcal pneumolysin (ply) (Kuo et al. (1995) Infect Immun 63:2706-2713) including ply detoxified in some fashion, for example dPLY-GMBS (WO 2004/081515, WO 2006/032499) or dPLY-formol, PhtX, including PhtA, PhtB, PhtD, PhtE (sequences of PhtA, PhtB, PhtD or PhtE are disclosed in WO 00/37105 and WO 00/39299) and fusions of Pht proteins, for example PhtDE fusions, PhtBE fusions, Pht A-E (WO 01/98334, WO 03/054007, WO 2009/000826), OMPC (meningococcal outer membrane protein), which is usually extracted from Neisseria meningitidis serogroup B (EP0372501), PorB (from N. meningitidis), PD (Haemophilus influenzae protein D; see, e.g., EP0594610 B), or immunologically functional equivalents thereof, synthetic peptides (EP0378881, EP0427347), heat shock proteins (WO 93/17712, WO 94/03208), pertussis proteins (WO 98/58668, EP0471177), cytokines, lymphokines, growth factors or hormones (WO 91/01146), artificial proteins comprising multiple human CD4+T cell epitopes from various pathogen derived antigens (Falugi et al. (2001) Eur J Immunol 31:3816-3824) such as N19 protein (Baraldoi et al. (2004) Infect Immun 72:4884-4887) pneumococcal surface protein PspA (WO 02/091998), iron uptake proteins (WO 01/72337), toxin A or B of Clostridium difficile (WO 00/61761), transferrin binding proteins, pneumococcal adhesion protein (PsaA), recombinant Pseudomonas aeruginosa exotoxin A (in particular non-toxic mutants thereof (such as exotoxin A bearing a substitution at glutamic acid 553 (Douglas et al. (1987) J. Bacteriol. 169 (11): 4967-4971)). Other proteins, such as ovalbumin, keyhole limpet hemocyanin (KLH), bovine serum albumin (BSA) or purified protein derivative of tuberculin (PPD) also can be used as carrier proteins. Other suitable carrier proteins include inactivated bacterial toxins such as cholera toxoid (e.g., as described in WO 2004/083251), Escherichia coli LT, E. coli ST, and exotoxin A from P. aeruginosa. Another suitable carrier protein is a C5a peptidase from Streptococcus (SCP).

In an embodiment, the carrier protein of the glycoconjugate of the invention is DT (Diphtheria toxoid). In another embodiment, the carrier protein of the glycoconjugate of the invention is TT (tetanus toxoid).

In another embodiment, the carrier protein of the glycoconjugate of the invention is PD (H. influenzae protein D; see, e.g., EP0594610 B).

In a preferred embodiment, the carrier protein of the glycoconjugate of the invention is TT, CRM₁₉₇ or a C5a peptidase from Streptococcus (SCP).

In a preferred embodiment, the carrier protein of the glycoconjugate of the invention is CRM197 or a C5a peptidase from Streptococcus (SCP).

In a very preferred embodiment, the carrier protein of the glycoconjugate of the invention is CRM₁₉₇. The CRM197 protein is a nontoxic form of diphtheria toxin but is immunologically indistinguishable from the diphtheria toxin. CRM197 is produced by Corynebacterium diphtheriae infected by the nontoxigenic phage β197tox-created by nitrosoguanidine mutagenesis of the toxigenic corynephage beta (Uchida et al. (1971) Nature New Biology 233:8-11). The CRM197 protein has the same molecular weight as the diphtheria toxin but differs therefrom by a single base change (guanine to adenine) in the structural gene. This single base change causes an amino acid substitution (glutamic acid for glycine) in the mature protein and eliminates the toxic properties of diphtheria toxin. The CRM197 protein is a safe and effective T-cell dependent carrier for saccharides. Further details about CRM197 and production thereof can be found, e.g., in U.S. Pat. No. 5,614,382.

In an embodiment, the carrier protein of the glycoconjugate of the invention is the A chain of CRM197 (see CN103495161). In an embodiment, the carrier protein of the glycoconjugate of the invention is the A chain of CRM197 obtained via expression by genetically recombinant E. coli (see CN103495161).

In an embodiment of any of the above vaccines, the capsular polysaccharides are all individually conjugated to CRM₁₉₇.

In an embodiment, the capsular polysaccharides from S. pneumoniae serotypes 1, 4, 5, 6B, 7F, 9V, 14 and/or 23F of any of the above vaccines are individually conjugated to PD.

In an embodiment, the capsular polysaccharide from S. pneumoniae serotype 18C of any of the above vaccine is conjugated to TT.

In an embodiment, the capsular polysaccharides from S. pneumoniae serotype 19F of any of the above vaccine is conjugated to DT.

In an embodiment, the capsular polysaccharides from S. pneumoniae serotypes 1, 4, 5, 6B, 7F, 9V, 14 and/or 23F of any of the above vaccine are individually conjugated to PD, the capsular polysaccharide from S. pneumoniae serotype 18C is conjugated to TT and the capsular polysaccharide from S. pneumoniae serotype 19F is conjugated to DT.

In an embodiment of any of the above vaccine, at least one capsular polysaccharide is conjugated to TT and the other capsular polysaccharides are all conjugated to CRM₁₉₇.

In an embodiment of any of the above vaccine, one capsular polysaccharide is conjugated to TT and the other capsular polysaccharides are all conjugated to CRM₁₉₇.

In an embodiment of any of the above vaccine, at least two capsular polysaccharides are conjugated to TT and the other capsular polysaccharides are all conjugated to CRM₁₉₇.

In an embodiment of any of the above vaccine, two capsular polysaccharides are conjugated to TT and the other capsular polysaccharides are all conjugated to CRM₁₉₇.

In an embodiment of any of the above vaccine, at least three capsular polysaccharides are conjugated to TT and the other capsular polysaccharides are all conjugated to CRM₁₉₇.

In an embodiment of any of the above vaccine, three capsular polysaccharides are conjugated to TT and the other capsular polysaccharides are all conjugated to CRM₁₉₇.

In an embodiment of any of the above vaccine, four capsular polysaccharides are conjugated to TT and the other capsular polysaccharides are all conjugated to CRM₁₉₇.

In an embodiment the pneumococcal conjugate vaccine of the invention is a 21-valent pneumococcal conjugate wherein said 21 glycoconjugates are from S. pneumoniae serotypes 1, 3, 4, 5, 6A, 6B, 7F, 8, 9N, 9V, 10A, 11A, 12F, 14, 15B, 18C, 19A, 19F, 22F, 23F and 33F, wherein four capsular polysaccharides are conjugated to TT wherein the four capsular polysaccharides that are conjugated to TT are serotypes 15B, 22F and the other two serotypes are selected from the group consisting of serotypes 1, 3 and 5 and the other glycoconjugates are all conjugated to CRM₁₉₇.

In an embodiment the pneumococcal conjugate vaccine of the invention is a 21-valent pneumococcal conjugate wherein said 21 glycoconjugates are from S. pneumoniae serotypes 1, 3, 4, 5, 6A, 6B, 7F, 8, 9N, 9V, 10A, 11A, 12F, 14, 15B, 18C, 19A, 19F, 22F, 23F and 33F, wherein four capsular polysaccharides are conjugated to TT wherein the four capsular polysaccharides that are conjugated to TT are serotypes 1, 5, 15B and 22F and the other glycoconjugates are all conjugated to CRM₁₉₇.

In an embodiment the pneumococcal conjugate vaccine of the invention is a 21-valent pneumococcal conjugate wherein said 21 glycoconjugates are from S. pneumoniae serotypes 1, 3, 4, 5, 6A, 6B, 7F, 8, 9N, 9V, 10A, 11A, 12F, 14, 15B, 18C, 19A, 19F, 22F, 23F and 33F, wherein four capsular polysaccharides are conjugated to TT wherein the four capsular polysaccharides that are conjugated to TT are serotypes 1, 3, 15B and 22F and the other glycoconjugates are all conjugated to CRM₁₉₇.

In an embodiment the pneumococcal conjugate vaccine of the invention is a 21-valent pneumococcal conjugate wherein said 21 glycoconjugates are from S. pneumoniae serotypes 1, 3, 4, 5, 6A, 6B, 7F, 8, 9N, 9V, 10A, 11A, 12F, 14, 15B, 18C, 19A, 19F, 22F, 23F and 33F, wherein four capsular polysaccharides are conjugated to TT wherein the four capsular polysaccharides that are conjugated to TT are serotypes 3, 5, 15B and 22F and the other glycoconjugates are all conjugated to CRM₁₉₇.

In an embodiment the pneumococcal conjugate vaccine of the invention is a 21-valent pneumococcal conjugate wherein said 21 glycoconjugates are from S. pneumoniae serotypes 1, 3, 4, 5, 6A, 6B, 7F, 8, 9N, 9V, 10A, 11A, 12F, 14, 15B, 18C, 19A, 19F, 22F, 23F and 33F, wherein two glycoconjugates selected from serotypes 1, 3 and 5 are conjugated to TT and the other glycoconjugates are all conjugated to CRM₁₉₇.

In an embodiment the pneumococcal conjugate vaccine of the invention is a 21-valent pneumococcal conjugate wherein said 21 glycoconjugates are from S. pneumoniae serotypes 1, 3, 4, 5, 6A, 6B, 7F, 8, 9N, 9V, 10A, 11A, 12F, 14, 15B, 18C, 19A, 19F, 22F, 23F and 33F, wherein glycoconjugates from serotypes 1 and 3 are conjugated to TT and the other glycoconjugates are all conjugated to CRM₁₉₇.

In an embodiment the pneumococcal conjugate vaccine of the invention is a 21-valent pneumococcal conjugate wherein said 21 glycoconjugates are from S. pneumoniae serotypes 1, 3, 4, 5, 6A, 6B, 7F, 8, 9N, 9V, 10A, 11A, 12F, 14, 15B, 18C, 19A, 19F, 22F, 23F and 33F, wherein glycoconjugates from serotypes 1 and 5 are conjugated to TT and the other glycoconjugates are all conjugated to CRM₁₉₇.

In an embodiment the pneumococcal conjugate vaccine of the invention is a 21-valent pneumococcal conjugate wherein said 21 glycoconjugates are from S. pneumoniae serotypes 1, 3, 4, 5, 6A, 6B, 7F, 8, 9N, 9V, 10A, 11A, 12F, 14, 15B, 18C, 19A, 19F, 22F, 23F and 33F, wherein glycoconjugates from serotypes 3 and 5 are conjugated to TT and the other glycoconjugates are all conjugated to CRM₁₉₇.

In an embodiment the pneumococcal conjugate vaccine of the invention is a 15-valent pneumococcal conjugate wherein said 15 glycoconjugates are from S. pneumoniae serotypes 1, 3, 4, 5, 6A, 6B, 7F, 9V, 14, 18C, 19A, 19F, 22F, 23F and 33F, wherein all the glycoconjugates are conjugated to CRM₁₉₇.

In a preferred embodiment the pneumococcal conjugate vaccine of the invention is a 20-valent pneumococcal conjugate wherein said 20 glycoconjugates are from S. pneumoniae serotypes 1, 3, 4, 5, 6A, 6B, 7F, 8, 9V, 10A, 11A, 12F, 14, 15B, 18C, 19A, 19F, 22F, 23F and 33F, wherein all the glycoconjugates are conjugated to CRM₁₉₇.

Vaccines of the invention may include a small amount of free carrier. When a given carrier protein is present in both free and conjugated form in a vaccine of the invention, the unconjugated form is preferably no more than 5% of the total amount of the carrier protein in the composition as a whole, and more preferably present at less than 2% by weight.

In some embodiments, the vaccine of the invention may comprise at least one adjuvant. In some embodiments, the vaccine of the invention may comprise one adjuvant. The term “adjuvant” refers to a compound or mixture that enhances the immune response to an antigen. Antigens may act primarily as a delivery system, primarily as an immune modulator or have strong features of both.

Suitable adjuvants include those suitable for use in mammals, including humans. Examples of known suitable delivery-system type adjuvants that can be used in humans include, but are not limited to, alum (e.g., aluminum phosphate, aluminum sulfate or aluminum hydroxide), calcium phosphate, liposomes, oil-in-water emulsions such as MF59 (4.3% w/v squalene, 0.5% w/v polysorbate 80 (Tween 80), 0.5% w/v sorbitan trioleate (Span 85)), water-in-oil emulsions such as Montanide, and poly(D,L-lactide-co-glycolide) (PLG) microparticles or nanoparticles.

In an embodiment, the vaccine of the invention comprises aluminum salts (alum) as adjuvant (e.g., aluminum phosphate, aluminum sulfate or aluminum hydroxide). In a preferred embodiment, the vaccine of the invention comprises aluminum phosphate or aluminum hydroxide as adjuvant. In an even preferred embodiment, the vaccine of the invention comprises aluminum phosphate as adjuvant.

In some embodiments, the vaccine of the disclosure comprises aluminum phosphate as adjuvant and has a final aluminum phosphate concentration between about 0.1 mg/mL and about 1 mg/ml. In some embodiments, the vaccine of the disclosure comprises aluminum phosphate as adjuvant and has a final aluminum phosphate concentration between about 0.1 mg/ml and about 0.5 mg/ml. In a preferred embodiment, the vaccine of the disclosure comprises aluminum phosphate as adjuvant and has a final aluminum phosphate concentration of about 0.25 mg/ml.

In an embodiment, the adjuvant is added to the vaccine after the addition of the preservative. In a preferred embodiment, the adjuvant is added to the vaccine after the addition of a 2-PE preservative. In another preferred embodiment, an aluminum phosphate adjuvant is added to the vaccine after the addition of a 2-PE preservative.

Further exemplary adjuvants to enhance effectiveness of the vaccine of the invention includes, but are not limited to: (1) oil-in-water emulsion formulations (with or without other specific immunostimulating agents such as muramyl peptides (see below) or bacterial cell wall components), such as for example (a) SAF, containing 10% Squalene, 0.4% Tween 80, 5% pluronic-blocked polymer L121, and thr-MDP either microfluidized into a submicron emulsion or vortexed to generate a larger particle size emulsion, and (b) RIBI™ adjuvant system (RAS), (Ribi Immunochem, Hamilton, MT) containing 2% Squalene, 0.2% Tween 80, and one or more bacterial cell wall components such as monophosphorylipid A (MPL), trehalose dimycolate (TDM), and cell wall skeleton (CWS), preferably MPL+CWS (DETOX™); (2) saponin adjuvants, such as QS21, STIMULON™ (Cambridge Bioscience, Worcester, MA), ABISCO® (Isconova, Sweden), or ISCOMATRIX® (Commonwealth Serum Laboratories, Australia), may be used or particles generated therefrom such as ISCOMs (immunostimulating complexes), which ISCOMS may be devoid of additional detergent (e.g., WO 00/07621); (3) Complete Freund's Adjuvant (CFA) and Incomplete Freund's Adjuvant (IFA); (4) cytokines, such as interleukins (e.g., IL-1, IL-2, IL-4, IL-5, IL-6, IL-7, IL-12 (e.g., WO 99/44636)), interferons (e.g., gamma interferon), macrophage colony stimulating factor (M-CSF), tumor necrosis factor (TNF), etc.; (5) monophosphoryl lipid A (MPL) or 3-O-deacylated MPL (3dMPL) (see, e.g., GB-2220221, EP0689454), optionally in the substantial absence of alum when used with pneumococcal saccharides (see, e.g., WO 00/56358); (6) combinations of 3dMPL with, for example, QS21 and/or oil-in-water emulsions (see, e.g., EP0835318, EP0735898, EP0761231); (7) a polyoxyethylene ether or a polyoxyethylene ester (see, e.g., WO 99/52549); (8) a polyoxyethylene sorbitan ester surfactant in combination with an octoxynol (e.g., WO 01/21207) or a polyoxyethylene alkyl ether or ester surfactant in combination with at least one additional non-ionic surfactant such as an octoxynol (e.g., WO 01/21152); (9) a saponin and an immunostimulatory oligonucleotide (e.g., a CpG oligonucleotide) (e.g., WO 00/62800); (10) an immunostimulant and a particle of metal salt (see, e.g., WO 00/23105); (11) a saponin and an oil-in-water emulsion (e.g., WO 99/11241); (12) a saponin (e.g., QS21)+3dMPL+IM2 (optionally+a sterol) (e.g., WO 98/57659); (13) other substances that act as immunostimulating agents to enhance the efficacy of the composition. Muramyl peptides include N-acetyl-muramyl-L-threonyl-D-isoglutamine (thr-MDP), N-25 acetyl-normuramyl-L-alanyl-D-isoglutamine (nor-MDP), N-acetylmuramyl-L-alanyl-D-isoglutarninyl-L-alanine-2-(1′-2′-dipalmitoyl-sn-glycero-3-hydroxyphosphoryloxy)-ethylamine MTP-PE), etc.

In an embodiment of the present invention, the vaccine of the invention comprises a CpG Oligonucleotide as adjuvant. A CpG oligonucleotide as used herein refers to an immunostimulatory CpG oligodeoxynucleotide (CpG ODN), and accordingly these terms are used interchangeably unless otherwise indicated. Immunostimulatory CpG oligodeoxynucleotides contain one or more immunostimulatory CpG motifs that are unmethylated cytosine-guanine dinucleotides, optionally within certain preferred base contexts. The methylation status of the CpG immunostimulatory motif generally refers to the cytosine residue in the dinucleotide. An immunostimulatory oligonucleotide containing at least one unmethylated CpG dinucleotide is an oligonucleotide which contains a 5′ unmethylated cytosine linked by a phosphate bond to a 3′ guanine, and which activates the immune system through binding to Toll-like receptor 9 (TLR-9). In another embodiment the immunostimulatory oligonucleotide may contain one or more methylated CpG dinucleotides, which will activate the immune system through TLR9 but not as strongly as if the CpG motif(s) was/were unmethylated. CpG immunostimulatory oligonucleotides may comprise one or more palindromes that in turn may encompass the CpG dinucleotide. CpG oligonucleotides have been described in a number of issued patents, published patent applications, and other publications, including U.S. Pat. Nos. 6,194,388; 6,207,646; 6,214,806; 6,218,371; 6,239,116; and 6,339,068.

In an embodiment of the present invention, the vaccine of the invention comprises any of the CpG Oligonucleotide described at page 3, line 22, to page 12, line 36, of WO 2010/125480.

Different classes of CpG immunostimulatory oligonucleotides have been identified. These are referred to as A, B, C and P class, and are described in greater detail at page 3, line 22, to page 12, line 36, of WO 2010/125480. Methods of the invention embrace the use of these different classes of CpG immunostimulatory oligonucleotides.

In an embodiment, the vaccine of the invention comprises any of the combination of glycoconjugate(s) disclosed herein and a pharmaceutically acceptable excipient, carrier, or diluent.

In addition to the conjugate(s) and the preservative, the vaccines of the disclosure may comprise one or more of a buffer, a salt, a divalent cation, a non-ionic detergent, a cryoprotectant such as a sugar, and an anti-oxidant such as a free radical scavenger or chelating agent, or any multiple combinations thereof.

In an embodiment, the vaccines of the disclosure comprise a buffer. In an embodiment, said buffer has a pKa of about 3.5 to about 7.5. In some embodiments, the buffer is phosphate, succinate, histidine or citrate. In some embodiments, the buffer is succinate. In some embodiments, the buffer is histidine. In certain embodiments, the buffer is succinate at a final concentration of 1 mM to 10 mM. In one particular embodiment, the final concentration of the succinate buffer is about 5 mM.

In an embodiment, the vaccines of the disclosure comprise a salt. In some embodiments, the salt is selected from the groups consisting of magnesium chloride, potassium chloride, sodium chloride and a combination thereof. In one particular embodiment, the salt is sodium chloride. In one particular embodiment, the immunogenic compositions of the invention comprise sodium chloride at 150 mM.

In an embodiment, the vaccines of the disclosure comprise a surfactant. In an embodiment, the surfactant is selected from the group consisting of polysorbate 20 (TWEEN™20), polysorbate 40 (TWEEN™40), polysorbate 60 (TWEEN™60), polysorbate 65 (TWEEN™65), polysorbate 80 (TWEEN™80), polysorbate 85 (TWEEN™85), TRITON™ N-101, TRITON™ X-100, oxtoxynol 40, nonoxynol-9, triethanolamine, triethanolamine polypeptide oleate, polyoxyethylene-660 hydroxystearate (PEG-15, Solutol H 15), polyoxyethylene-35-ricinoleate (CREMOPHOR® EL), soy lecithin and a poloxamer.

In one particular embodiment, the surfactant is polysorbate 80. In some said embodiment, the final concentration of polysorbate 80 in the formulation is at least 0.0001% to 10% polysorbate 80 weight to weight (w/w). In some said embodiments, the final concentration of polysorbate 80 in the formulation is at least 0.001% to 1% polysorbate 80 weight to weight (w/w). In some said embodiments, the final concentration of polysorbate 80 in the formulation is at least 0.01% to 1% polysorbate 80 weight to weight (w/w). In other embodiments, the final concentration of polysorbate 80 in the formulation is 0.01%, 0.02%, 0.03%, 0.04%, 0.05%, 0.06%, 0.07%, 0.08%, 0.09% or 0.1% polysorbate 80 (w/w). In a preferred embodiment, the final concentration of polysorbate 80 in the formulation is between 0.01% and 0.03%. In another embodiment, the final concentration of the polysorbate 80 in the formulation is 0.02% polysorbate 80 (w/w). In another embodiment, the final concentration of the polysorbate 80 in the formulation is 0.01% polysorbate 80 (w/w). In another embodiment, the final concentration of the polysorbate 80 in the formulation is 0.03% polysorbate 80 (w/w). In another embodiment, the final concentration of the polysorbate 80 in the formulation is 0.04% polysorbate 80 (w/w). In another embodiment, the final concentration of the polysorbate 80 in the formulation is 0.05% polysorbate 80 (w/w). In another embodiment, the final concentration of the polysorbate 80 in the formulation is 1% polysorbate 80 (w/w).

In one particular embodiment, the surfactant is polysorbate 20. In some said embodiment, the final concentration of polysorbate 20 in the formulation is at least 0.01% to 10% polysorbate 20 weight to weight (w/w). In some said embodiments, the final concentration of polysorbate 20 in the formulation is at least 0.1% to 1% polysorbate 20 weight to weight (w/w). In some said embodiments, the final concentration of polysorbate 20 in the formulation is at least 0.1% to 0.5% polysorbate 20 weight to weight (w/w). In other embodiments, the final concentration of polysorbate 20 in the formulation is 0.1%, 0.2%, 0.3%, 0.4%, 0.5%, 0.6%, 0.7%, 0.8%, 0.9% or 1% polysorbate 20 (w/w). In another embodiment, the final concentration of the polysorbate 20 in the formulation is 0.2% polysorbate 20 (w/w). In another embodiment, the final concentration of the polysorbate 20 in the formulation is 0.1% polysorbate 20 (w/w). In another embodiment, the final concentration of the polysorbate 20 in the formulation is 0.3% polysorbate 20 (w/w). In another embodiment, the final concentration of the polysorbate 20 in the formulation is 0.4% polysorbate 80 (w/w). In another embodiment, the final concentration of the polysorbate 20 in the formulation is 0.5% polysorbate 20 (w/w). In a preferred embodiment, the final concentration of the polysorbate 20 in the formulation is 0.2% polysorbate 20 (w/w).

In one particular embodiment, the surfactant is polysorbate 40. In some said embodiment, the final concentration of polysorbate 40 in the formulation is at least 0.0001% to 10% polysorbate 40 weight to weight (w/w). In some said embodiments, the final concentration of polysorbate 40 in the formulation is at least 0.001% to 1% polysorbate 40 weight to weight (w/w). In some said embodiments, the final concentration of polysorbate 40 in the formulation is at least 0.01% to 1% polysorbate 40 weight to weight (w/w). In other embodiments, the final concentration of polysorbate 40 in the formulation is 0.01%, 0.02%, 0.03%, 0.04%, 0.05%, 0.06%, 0.07%, 0.08%, 0.09% or 0.1% polysorbate 40 (w/w). In another embodiment, the final concentration of the polysorbate 40 in the formulation is 1% polysorbate 40 (w/w).

In one particular embodiment, the surfactant is polysorbate 60. In some said embodiment, the final concentration of polysorbate 60 in the formulation is at least 0.0001% to 10% polysorbate 60 weight to weight (w/w). In some said embodiments, the final concentration of polysorbate 60 in the formulation is at least 0.001% to 1% polysorbate 60 weight to weight (w/w). In some said embodiments, the final concentration of polysorbate 60 in the formulation is at least 0.01% to 1% polysorbate 60 weight to weight (w/w). In other embodiments, the final concentration of polysorbate 60 in the formulation is 0.01%, 0.02%, 0.03%, 0.04%, 0.05%, 0.06%, 0.07%, 0.08%, 0.09% or 0.1% polysorbate 60 (w/w). In another embodiment, the final concentration of the polysorbate 60 in the formulation is 1% polysorbate 60 (w/w).

In one particular embodiment, the surfactant is polysorbate 65. In some said embodiment, the final concentration of polysorbate 65 in the formulation is at least 0.0001% to 10% polysorbate 65 weight to weight (w/w). In some said embodiments, the final concentration of polysorbate 65 in the formulation is at least 0.001% to 1% polysorbate 65 weight to weight (w/w). In some said embodiments, the final concentration of polysorbate 65 in the formulation is at least 0.01% to 1% polysorbate 65 weight to weight (w/w). In other embodiments, the final concentration of polysorbate 65 in the formulation is 0.01%, 0.02%, 0.03%, 0.04%, 0.05%, 0.06%, 0.07%, 0.08%, 0.09% or 0.1% polysorbate 65 (w/w). In another embodiment, the final concentration of the polysorbate 65 in the formulation is 1% polysorbate 65 (w/w).

In one particular embodiment, the surfactant is polysorbate 85. In some said embodiment, the final concentration of polysorbate 85 in the formulation is at least 0.0001% to 10% polysorbate 85 weight to weight (w/w). In some said embodiments, the final concentration of polysorbate 85 in the formulation is at least 0.001% to 1% polysorbate 85 weight to weight (w/w). In some said embodiments, the final concentration of polysorbate 85 in the formulation is at least 0.01% to 1% polysorbate 85 weight to weight (w/w). In other embodiments, the final concentration of polysorbate 85 in the formulation is 0.01%, 0.02%, 0.03%, 0.04%, 0.05%, 0.06%, 0.07%, 0.08%, 0.09% or 0.1% polysorbate 85 (w/w). In another embodiment, the final concentration of the polysorbate 85 in the formulation is 1% polysorbate 85 (w/w).

In certain embodiments, the vaccine of the disclosure has a final pH of 5.5 to 7.5, more preferably a final pH of 5.6 to 7.0, even more preferably a final pH of 5.3 to 6.3. In a preferred embodiment, the vaccine of the disclosure has a final pH of 5.8 to 6.0. In another preferred embodiment, the vaccine of the disclosure has a final pH of 5.8.

In some embodiments, the vaccine of the disclosure has a final protein concentration between about 50 μg/ml and about 150 μg/ml. In a preferred embodiment, the vaccine of the disclosure has a final protein concentration between about 60 μg/ml and about 130 g/ml. In another preferred embodiment, the vaccine of the disclosure has a final protein concentration between about 90 μg/ml and about 99 μg/ml. In still another preferred embodiment, the vaccine of the disclosure has a final protein concentration of about 99 μg/ml.

In some embodiments, the vaccine of the disclosure has a final D10 particle size distribution between about 3,000 and about 6,000. In another embodiment, the vaccine of the disclosure has a final D10 particle size distribution between about 4,000 and about 5,000. In a preferred embodiment, the vaccine of the disclosure has a final D10 particle size distribution between about 4,300 and about 4,700. In some embodiments, the vaccine of the disclosure has a final D50 particle size distribution between about 6,000 and about 8,000. In another embodiment, the vaccine of the disclosure has a final D50 particle size distribution between about 7,000 and about 8,000. In a preferred embodiment, the vaccine of the disclosure has a final D50 particle size distribution between about 7,500 and about 7,900. In some embodiments, the vaccine of the disclosure has a final D90 particle size distribution between about 10,000 and about 16,000. In another embodiment, the vaccine of the disclosure has a final D90 particle size distribution between about 12,000 and about 15,000. In a preferred embodiment, the vaccine of the disclosure has a final D90 particle size distribution between about 12,300 and about 14,900.

A typical dose of the vaccine of the invention for injection has a volume of 0.1 mL to 2 mL. In an embodiment, the vaccine of the invention for injection has a volume of 0.2 mL to 1 mL, even more preferably a volume of about 0.5 mL. Most preferably, the vaccine of the invention for injection has a volume of 0.5 mL.

The amount of glycoconjugate(s) in each dose is selected as an amount which induces an immunoprotective response without significant, adverse side effects in typical vaccines. Such amount will vary depending upon which specific immunogen is employed and how it is presented.

The amount of a particular glycoconjugate in a vaccine can be calculated based on total polysaccharide for that conjugate (conjugated and non-conjugated). For example, a glycoconjugate with 20% free polysaccharide will have about 80 μg of conjugated polysaccharide and about 20 μg of nonconjugated polysaccharide in a 100 μg polysaccharide dose. The amount of glycoconjugate can vary depending upon the pneumococcal serotype. The saccharide concentration can be determined by the uronic acid assay.

The “immunogenic amount” of the different polysaccharide components in the vaccine, may diverge. Generally, each dose will comprise 0.1 μg to 100 μg of polysaccharide for a given serotype. In an embodiment each dose will comprise 0.1 μg to 100 μg of polysaccharide for a given serotype. In a preferred embodiment each dose will comprise 0.5 μg to 20 μg. In a preferred embodiment each dose will comprise 1.0 μg to 10 μg. In an even preferred embodiment, each dose will comprise 2.0 μg to 5.0 μg of polysaccharide for a given serotype. Any whole number integer within any of the above ranges is contemplated as an embodiment of the disclosure.

In an embodiment, each dose will comprise about 1.1 μg, about 1.2 μg, about 1.3 μg, about 1.4 μg, about 1.5 μg, about 1.6 μg, about 1.7 μg, about 1.8 μg, about 1.9 μg, about 2.0 μg, about 2.1 μg, about 2.2 μg, about 2.3 μg, about 2.4 μg, about 2.5 μg, about 2.6 μg, about 2.7 μg, about 2.8 μg, about 2.9 μg, or about 3.0 μg of polysaccharide for glycoconjugates from S. pneumoniae serotype 1, 3, 4, 5, 6A, 7F, 8, 9V, 10A, 11A, 12F, 14, 15B, 18C, 19A, 19F, 22F, 23F and/or 33F.

In an embodiment, each dose will comprise about 2.0 μg of polysaccharide for glycoconjugates from S. pneumoniae serotype 1, 3, 4, 5, 6A, 7F, 8, 9V, 10A, 11A, 12F, 14, 15B, 18C, 19A, 19F, 22F, 23F and/or 33F.

In an embodiment, each dose will comprise about 2.2 μg of polysaccharide for glycoconjugates from S. pneumoniae serotype 1, 3, 4, 5, 6A, 7F, 8, 9V, 10A, 11A, 12F, 14, 15B, 18C, 19A, 19F, 22F, 23F and/or 33F.

In an embodiment, each dose will comprise about 4.0 μg of polysaccharide for glycoconjugates from S. pneumoniae serotype 6B.

In an embodiment, each dose will comprise about 4.4 μg of polysaccharide for glycoconjugates from S. pneumoniae serotype 6B.

In an embodiment, each dose will comprise about 1.5 μg to about 3.0 μg of polysaccharide for each glycoconjugate from S. pneumoniae serotype 1, 3, 4, 5, 6A, 7F, 8, 9V, 10A, 11A, 12F, 14, 15B, 18C, 19A, 19F, 22F, 23F and 33F, and about 3.0 μg to about 6.0 μg of polysaccharide for glycoconjugate from S. pneumoniae serotype 6B.

In an embodiment, each dose will comprise about 2.0 μg to about 2.5 μg of polysaccharide for each glycoconjugate from S. pneumoniae serotype 1, 3, 4, 5, 6A, 7F, 8, 9V, 10A, 11A, 12F, 14, 15B, 18C, 19A, 19F, 22F, 23F and 33F, and about 4.0 μg to about 4.8 μg of polysaccharide for glycoconjugate from S. pneumoniae serotype 6B.

In an embodiment, each dose will comprise about 2.2 μg of polysaccharide from each glycoconjugate from S. pneumoniae serotype 1, 3, 4, 5, 6A, 7F, 8, 9V, 10A, 11A, 12F, 14, 15B, 18C, 19A, 19F, 22F, 23F and 33F, and about 4.4 μg of polysaccharide for glycoconjugate from S. pneumoniae serotype 6B.

In an embodiment, each dose will comprise about 1.5 μg to about 3.0 μg of polysaccharide for each glycoconjugate from S. pneumoniae serotype 1, 3, 4, 5, 6A, 7F, 9V, 14, 18C, 19A, 19F, 22F, 23F and 33F, and about 3 μg to about 6 μg of polysaccharide for glycoconjugate from S. pneumoniae serotype 6B.

In an embodiment, each dose will comprise about 2.0 μg to about 2.5 μg of polysaccharide for each glycoconjugate from S. pneumoniae serotype 1, 3, 4, 5, 6A, 7F, 9V, 14, 18C, 19A, 19F, 22F, 23F and 33F, and about 4.0 μg to about 4.8 μg of polysaccharide for glycoconjugate from S. pneumoniae serotype 6B.

In an embodiment, each dose will comprise about 2.0 μg of polysaccharide from each glycoconjugate from S. pneumoniae serotype 1, 3, 4, 5, 6A, 7F, 9V, 14, 18C, 19A, 19F, 22F, 23F and 33F, and about 4.0 μg of polysaccharide for glycoconjugate from S. pneumoniae serotype 6B.

In an embodiment, each dose will comprise about 1.5 μg to about 3.0 μg of polysaccharide for each glycoconjugate from S. pneumoniae serotype 1, 3, 4, 5, 6A, 7F, 9N, 9V, 14, 18C, 19A, 19F, 22F, 23F and 33F, and about 3 μg to about 6 μg of polysaccharide for glycoconjugate from S. pneumoniae serotype 6B.

In an embodiment, each dose will comprise about 2.0 μg to about 2.5 μg of polysaccharide for each glycoconjugate from S. pneumoniae serotype 1, 3, 4, 5, 6A, 7F, 9N, 9V, 14, 18C, 19A, 19F, 22F, 23F and 33F, and about 4.0 μg to about 4.8 μg of polysaccharide for glycoconjugate from S. pneumoniae serotype 6B.

In an embodiment, each dose will comprise about 2.0 μg of polysaccharide from each glycoconjugate from S. pneumoniae serotype 1, 3, 4, 5, 6A, 7F, 9N, 9V, 14, 18C, 19A, 19F, 22F, 23F and 33F, and about 4.0 μg of polysaccharide for glycoconjugate from S. pneumoniae serotype 6B.

Generally, each dose will comprise a total amount of 10 μg to 150 μg of carrier protein. In an embodiment, each dose will comprise a total amount of 10 μg to 150 μg of carrier protein. In an embodiment, each dose will comprise a total amount of 25 μg to 75 μg of carrier protein. In a preferred embodiment, each dose will comprise a total amount 40 μg to 60 μg of carrier protein. In an embodiment, said carrier protein is CRM₁₉₇.

In an embodiment, each dose will comprise about 30 μg of carrier protein. In an embodiment, each dose will comprise about 31 μg of carrier protein. In an embodiment, each dose will comprise about 32 μg of carrier protein. In an embodiment, each dose will comprise about 33 μg of carrier protein. In a preferred embodiment, each dose will comprise about 34 μg of carrier protein.

In a preferred embodiment, the vaccine is a 13-valent pneumococcal conjugate vaccine and each dose comprises about 34 μg of carrier protein. In an embodiment, said carrier protein is CRM₁₉₇.

In another preferred embodiment, the vaccine is a 15-valent pneumococcal conjugate vaccine and each dose comprises about 30 μg of carrier protein. In an embodiment, said carrier protein is CRM₁₉₇.

In an embodiment, each dose will comprise about 40 μg of carrier protein. In an embodiment, each dose will comprise about 41 μg of carrier protein. In an embodiment, each dose will comprise about 42 μg of carrier protein. In an embodiment, each dose will comprise about 43 μg of carrier protein. In an embodiment, each dose will comprise about 44 μg of carrier protein. In an embodiment, each dose will comprise about 45 μg of carrier protein.

In a preferred embodiment, the vaccine is a 16-valent pneumococcal conjugate vaccine and each dose comprises about 42 μg of carrier protein. In an embodiment, said carrier protein is CRM₁₉₇.

In an embodiment, each dose will comprise about 48 μg of carrier protein. In an embodiment, each dose will comprise about 49 μg of carrier protein. In an embodiment, each dose will comprise about 50 μg of carrier protein. In an embodiment, each dose will comprise about 51 μg of carrier protein. In an embodiment, each dose will comprise about 52 μg of carrier protein. In an embodiment, each dose will comprise about 53 μg of carrier protein.

In a preferred embodiment, the vaccine is a 20-valent pneumococcal conjugate vaccine and each dose comprises about 50 μg of carrier protein. In an embodiment, said carrier protein is CRM₁₉₇.

In a preferred embodiment the pneumococcal conjugate vaccine of the invention is a 20-valent pneumococcal conjugate vaccine wherein said 20 glycoconjugates are from S. pneumoniae serotypes 1, 3, 4, 5, 6A, 6B, 7F, 8, 9V, 10A, 11A, 12F, 14, 15B, 18C, 19A, 19F, 22F, 23F and 33F, wherein the glycoconjugates are all conjugated to CRM₁₉₇, and each dose comprises about 2.2 μg of each of capsular polysaccharides serotypes 1, 3, 4, 5, 6A, 7F, 8, 9V, 10A, 11A, 12F, 14, 15B, 18C, 19A, 19F, 22F, 23F, and 33F and about 4.4 μg of the capsular polysaccharide of serotype 6B, about 50 μg of CRM₁₉₇, 2-PE, 0.125 mg elemental aluminum as aluminum phosphate adjuvant, 100 μg polysorbate 80, sodium chloride and a succinate buffer.

In an embodiment the pneumococcal conjugate vaccine of the invention is a 15-valent pneumococcal conjugate vaccine wherein said 15 glycoconjugates are from S. pneumoniae serotypes 1, 3, 4, 5, 6A, 6B, 7F, 9V, 14, 18C, 19A, 19F, 22F, 23F and 33F, wherein the glycoconjugates are all conjugated to CRM197, and each dose comprises about 2.0 μg of each of capsular polysaccharides serotypes 1, 3, 4, 5, 6A, 7F, 9V, 14, 18C, 19A, 19F, 22F, 23F, and 33F and about 4.0 μg of the capsular polysaccharide of serotype 6B, about 30 μg of CRM197, 2-PE, 0.125 mg elemental aluminum as aluminum phosphate adjuvant, sodium chloride and a L-Histidine buffer.

In an embodiment the pneumococcal conjugate vaccine of the invention is a 15-valent pneumococcal conjugate vaccine wherein said 15 glycoconjugates are from S. pneumoniae serotypes 1, 3, 4, 5, 6A, 6B, 7F, 9V, 14, 18C, 19A, 19F, 22F, 23F and 33F, wherein the glycoconjugates are all conjugated to CRM197, and each dose comprises about 2.0 μg of each of capsular polysaccharides serotypes 1, 3, 4, 5, 6A, 7F, 9V, 14, 18C, 19A, 19F, 22F, 23F, and 33F and about 4.0 μg of the capsular polysaccharide of serotype 6B, about 30 μg of CRM197, 2-PE, 0.125 mg elemental aluminum as aluminum phosphate adjuvant, polysorbate 20, sodium chloride and a L-Histidine buffer.

In some embodiments, the vaccine is a 21-valent pneumococcal conjugate vaccine and each dose comprises about 1 μg to about 30 μg of TT and about 20 μg to about 85 μg of CRM₁₉₇.

In some embodiments, the vaccine is a 21-valent pneumococcal conjugate vaccine and each dose comprises about 2 μg to about 25 μg of TT and about 40 μg to about 75 μg of CRM₁₉₇.

In some embodiments, the vaccine is a 21-valent pneumococcal conjugate vaccine and each dose comprises about 2 μg to about 2.5 μg of each of capsular polysaccharides serotypes 1, 5, 6A, 7F, 8, 9N, 10A 11A, 12F, 14, 15B, 18C, 22F, 23F, and 33F and about 4 μg to about 5 μg of the capsular polysaccharides of serotypes 3, 4, 6B, 9V, 19A, and 19F.

In some embodiments, the vaccine is a 21-valent pneumococcal conjugate vaccine and each dose comprises about 2 μg to about 2.5 μg of each of capsular polysaccharides serotypes 1, 4, 5, 6A, 7F, 8, 9V, 9N, 10A, 11A, 12F, 14, 15B, 18C, 19A, 19F, 22F, 23F, and 33F and about 4 to about 5 μg of the capsular polysaccharides of serotypes 3 and 6B.

In an embodiment the pneumococcal conjugate vaccine of the invention is a 21-valent pneumococcal conjugate vaccine wherein said 21 glycoconjugates are from S. pneumoniae serotypes 1, 3, 4, 5, 6A, 6B, 7F, 8, 9N, 9V, 10A, 11A, 12F, 14, 15B, 18C, 19A, 19F, 22F, 23F and 33F, wherein glycoconjugates from serotypes 1 and 5 are conjugated to TT and the other glycoconjugates are all conjugated to CRM₁₉₇, and each dose comprises about 2 μg to about 2.5 μg of each of capsular polysaccharides serotypes 1, 3, 4, 5, 6A, 7F, 8, 9V, 9N, 10A, 11A, 12F, 14, 15B, 18C, 19A, 19F, 22F, 23F, and 33F and about 4 to about 5 μg of the capsular polysaccharide of serotype 6B.

In an embodiment the pneumococcal conjugate vaccine of the invention is a 21-valent pneumococcal conjugate vaccine wherein said 21 glycoconjugates are from S. pneumoniae serotypes 1, 3, 4, 5, 6A, 6B, 7F, 8, 9N, 9V, 10A, 11A, 12F, 14, 15B, 18C, 19A, 19F, 22F, 23F and 33F, wherein glycoconjugates from serotypes 1 and 5 are conjugated to TT and the other glycoconjugates are all conjugated to CRM₁₉₇, and each dose comprises about 2.2 μg of each of capsular polysaccharides serotypes 1, 3, 4, 5, 6A, 7F, 8, 9V, 9N, 10A, 11A, 12F, 14, 15B, 18C, 19A, 19F, 22F, 23F, and 33F and about 4.4 μg of the capsular polysaccharide of serotype 6B.

In an embodiment the pneumococcal conjugate vaccine of the invention is a 21-valent pneumococcal conjugate vaccine wherein said 21 glycoconjugates are from S. pneumoniae serotypes 1, 3, 4, 5, 6A, 6B, 7F, 8, 9N, 9V, 10A, 11A, 12F, 14, 15B, 18C, 19A, 19F, 22F, 23F and 33F, wherein glycoconjugates from serotypes 1 and 5 are conjugated to TT and the other glycoconjugates are all conjugated to CRM₁₉₇, and each dose comprises about 2 μg to about 2.5 μg of each of capsular polysaccharides serotypes 1, 5, 6A, 7F, 8, 9N, 10A, 11A, 12F, 14, 15B, 18C, 22F, 23F, and 33F and about 4 to about 5 μg of each of capsular polysaccharides serotypes 3, 4, 6B, 9V, 19A and 19F.

In an embodiment the pneumococcal conjugate vaccine of the invention is a 21-valent pneumococcal conjugate vaccine wherein said 21 glycoconjugates are from S. pneumoniae serotypes 1, 3, 4, 5, 6A, 6B, 7F, 8, 9N, 9V, 10A, 11A, 12F, 14, 15B, 18C, 19A, 19F, 22F, 23F and 33F, wherein glycoconjugates from serotypes 1 and 5 are conjugated to TT and the other glycoconjugates are all conjugated to CRM₁₉₇, and each dose comprises about 2.2 μg of each of capsular polysaccharides serotypes 1, 5, 6A, 7F, 8, 9N, 10A, 11A, 12F, 14, 15B, 18C, 22F, 23F, and 33F and about 4.4 μg of each of capsular polysaccharides serotypes 3, 4, 6B, 9V, 19A and 19F.

In an embodiment the pneumococcal conjugate vaccine of the invention is a 21-valent pneumococcal conjugate vaccine wherein said 21 glycoconjugates are from S. pneumoniae serotypes 1, 3, 4, 5, 6A, 6B, 7F, 8, 9N, 9V, 10A, 11A, 12F, 14, 15B, 18C, 19A, 19F, 22F, 23F and 33F, wherein glycoconjugates from serotypes 1 and 5 are conjugated to TT and the other glycoconjugates are all conjugated to CRM₁₉₇, and each dose comprises about 2 μg to about 2.5 μg of each of capsular polysaccharides serotypes 1, 3, 4, 5, 6A, 7F, 8, 9V, 9N, 10A, 11A, 12F, 14, 15B, 18C, 19A, 19F, 22F, 23F, and 33F and about 4 to about 5 μg of the capsular polysaccharide of serotype 6B, about 2 μg to about 25 μg of TT and about 40 μg to about 75 μg of CRM₁₉₇.

In an embodiment the pneumococcal conjugate vaccine of the invention is a 21-valent pneumococcal conjugate vaccine wherein said 21 glycoconjugates are from S. pneumoniae serotypes 1, 3, 4, 5, 6A, 6B, 7F, 8, 9N, 9V, 10A, 11A, 12F, 14, 15B, 18C, 19A, 19F, 22F, 23F and 33F, wherein glycoconjugates from serotypes 1 and 5 are conjugated to TT and the other glycoconjugates are all conjugated to CRM₁₉₇, and each dose comprises about 2 μg to about 2.5 μg of each of capsular polysaccharides serotypes 1, 3, 4, 5, 6A, 7F, 8, 9V, 9N, 10A, 11A, 12F, 14, 15B, 18C, 19A, 19F, 22F, 23F, and 33F and about 4 to about 5 μg of the capsular polysaccharide of serotype 6B, about 2 μg to about 25 μg of TT, about 40 μg to about 75 μg of CRM₁₉₇, 2-PE, 0.125 to 0.250 mg elemental aluminum adjuvant, sodium chloride and a succinate buffer.

In an embodiment the pneumococcal conjugate vaccine of the invention is a 21-valent pneumococcal conjugate vaccine wherein said 21 glycoconjugates are from S. pneumoniae serotypes 1, 3, 4, 5, 6A, 6B, 7F, 8, 9N, 9V, 10A, 11A, 12F, 14, 15B, 18C, 19A, 19F, 22F, 23F and 33F, wherein glycoconjugates from serotypes 1 and 5 are conjugated to TT and the other glycoconjugates are all conjugated to CRM₁₉₇, and each dose comprises about 2.2 μg of each of capsular polysaccharides serotypes 1, 3, 4, 5, 6A, 7F, 8, 9V, 9N, 10A, 11A, 12F, 14, 15B, 18C, 19A, 19F, 22F, 23F, and 33F and about 4.4 μg of the capsular polysaccharide of serotype 6B, about 2 μg to about 25 μg of TT, about 40 μg to about 75 μg of CRM₁₉₇, 2-PE, 0.125 to 0.250 mg elemental aluminum adjuvant, sodium chloride and a succinate buffer.

In an embodiment the pneumococcal conjugate vaccine of the invention is a 21-valent pneumococcal conjugate vaccine wherein said 21 glycoconjugates are from S. pneumoniae serotypes 1, 3, 4, 5, 6A, 6B, 7F, 8, 9N, 9V, 10A, 11A, 12F, 14, 15B, 18C, 19A, 19F, 22F, 23F and 33F, wherein glycoconjugates from serotypes 1 and 5 are conjugated to TT and the other glycoconjugates are all conjugated to CRM₁₉₇, and each dose comprises about 2.2 μg of each of capsular polysaccharides serotypes 1, 3, 4, 5, 6A, 7F, 8, 9V, 9N, 10A, 11A, 12F, 14, 15B, 18C, 19A, 19F, 22F, 23F, and 33F and about 4.4 μg of the capsular polysaccharide of serotype 6B, about 2 μg to about 25 μg of TT, about 40 μg to about 75 μg of CRM₁₉₇, 2-PE, 0.125 to 0.250 mg elemental aluminum adjuvant, polysorbate 80, sodium chloride and a succinate buffer.

In a preferred embodiment, the dose of the vaccine of the present invention is a 0.5 mL dose.

As used herein, the term “about” means within a statistically meaningful range of a value, such as a stated amount of 2-PE, polysaccharide, carrier protein, concentration of buffer, volume or pH. Such a range can be within an order of magnitude, typically within 20%, more typically within 10%, and even more typically within 5% or within 1% of a given value or range. Sometimes, such a range can be within the experimental error typical of standard methods used for the measurement and/or determination of a given value or range. The allowable variation encompassed by the term “about” will depend upon the particular system under study, and can be readily appreciated by one of ordinary skill in the art. Whenever a range is recited within this application, every number within the range is also contemplated as an embodiment of the disclosure.

The terms “comprising”, “comprise” and “comprises” herein are intended by the inventors to be optionally substitutable with the terms “consisting essentially of”, “consist essentially of”, “consists essentially of”, “consisting of’, “consist of’ and “consists of’, respectively, in every instance.

An “immunogenic amount”, an “immunologically effective amount”, a “therapeutically effective amount”, a “prophylactically effective amount”, or “dose”, each of which is used interchangeably herein, generally refers to the amount of antigen or immunogenic composition sufficient to elicit an immune response, either a cellular (T cell) or humoral (B cell or antibody) response, or both, as measured by standard assays known to one skilled in the art.

Any whole number integer within any of the ranges of the present document is contemplated as an embodiment of the disclosure.

All references or patent applications cited within this patent specification are incorporated by reference herein.

The invention is illustrated in the accompanying examples. The examples below are carried out using standard techniques, which are well known and routine to those of skill in the art, except where otherwise described in detail. The examples are illustrative, but do not limit the invention.

### EXAMPLES

### Example 1 Solubility of 2-phenoxyethanol (2-PE)

The objective of this study was to evaluate the dissolution time and solubility of 2-phenoxyethanol (2-PE). 2-PE dissolution and concentration were evaluated at the following concentrations in Succinate Buffered Saline (SBS) (5 mM succinate, 150 mM NaCl, pH 5.8); 11 g/L, 16.5 g/L, and 20 g/L.

Appropriate amount of 2-PE was weighed out in 400 ml beaker and appropriate amount of succinate buffered saline was added. Agitation speed was 300 rpm and solution temperature about 20° C. The time for complete dissolution based on visual observation was recorded. 2-PE concentration was measured by RP-HPLC.

The dissolution time results are shown in Table 1 and 2-PE concentration results are shown in Table 2. The bulk dissolution time for 20 mg/mL 2-PE in succinate-buffered saline (15 minutes) was longer than 11 mg/ml (7 minutes) and 16.5 mg/ml (9 minutes) samples. A few small particles were noticed after completion of the first two dissolution tests (11 mg/mL and 16.5 mg/mL) but the complete dissolution was not tracked at the initial mixing speed (300 rpm). Based on these observations, the complete dissolution time was monitored for the 20 mg/ml sample. The complete dissolution (of all small particles) time was 35 minutes for the 20 mg/mL sample.

TABLE 1
Dissolution Time

| | 2-PE bulk | | |
|---|---|---|---|
| | dissolution | | 2-PE complete |
| Sample | Time* | | dissolution time |
| 11 mg/mL 2-PE (in SBS) | 7 | min | Not recorded |
| 16.5 mg/mL 2-PE (in SBS) | 9 | min | Agitation speed |
| | | | significantly increased |
| | | | after 9 min |
| 20 mg/mL 2-PE (in SBS) | 15 | min | 35 min |

*All 2-PE dissolved besides up to approximately 5 small particles

TABLE 2
2-PE concentration

| | Target 2-PE | Measured 2-PE | Percent |
|---|---|---|---|
| | concentration | concentration | Differ- |
| Sample | (mg/mL) | (mg/mL) | ence* |
| 11 mg/mL 2-PE (in SBS) | 11.0 | 10.5 | −4.5% |
| 16.5 mg/mL 2-PE (in SBS) | 16.5 | 15.6 | −5.5% |
| 20 mg/mL 2-PE (in SBS) | 20.0 | 19.1 | −4.5% |

*Calculation: % = (measured − target)/target × 100

The 2-PE concentration data demonstrate that 2-PE is soluble at 19.1 mg/ml or greater in succinate-buffered saline (pH 5.8) at 20° C.

The measured 2-PE concentration results were all approximately 5% below the target concentration. Since the percent different was consistent at all concentrations, a measured result of 5% below target for the 20 mg/ml sample is not expected to demonstrate reaching the solubility limit of 2-PE in succinate buffered saline (at 20° C.).

### Example 2 Impact to Filter Integrity of a Polyethersulfone (PES) Membrane (0.5 μm/0.2 μm) Filter When Exposed to Multivalent Conjugate Pool Comprising 2-Phenoxyethanol

The objective of this study was to assess the impact to filter integrity of PES membrane (0.5 μm/0.2 μm) filter when exposed for at least 48 hours to a 20-valent conjugate pool formulation. Testing has been performed on both the filter membrane and the filter device. The compatibility testing is also applicable to other filter sizes in the same filter family.

A 20-valent conjugate pool (conjugates of S. pneumoniae polysaccharide serotypes 1, 3, 4, 5, 6A, 6B, 7F, 8, 9V, 10A, 11A, 12F, 14, 15B, 18C, 19A, 19F, 22F, 23F and 33F conjugated to CRM₁₉₇ at a concentration of about 7.5 μg/ml each but for 6B at about 15 μg/ml, 5.5 mM Succinate Buffer pH 5.8, 0.033% (w/w) PS80, about 150 mM NaCl, see WO2015110941) was formulated with 17 mg/mL 2-phenoxyethanol (final concentration). The concentrations of conjugates are based on the saccharide content (anthrone saccharide concentration).

17 mg/ml of 2-PE in the conjugate pool equates to 10 mg/ml of 2-PE in the final drug product after final dilution with buffer and aluminum phosphate (AIPO4) addition.

Two capsule filters were thoroughly wetted by filtering through 1 L of deionized (DI) water for each filter. Both filters then underwent pre-use Filter Integrity Testing (FIT) at ambient temperature (22±4° C.). After both filters passed pre-use FIT, the conjugate pool material containing 17 mg/mL 2-PE was filled into each filter device. The conjugate pool material underwent a static hold in each filter device for a minimum 48 hours at 22±4° C. After 48 hours, the conjugate pool material was drained from the filter devices then flushed with 2 L of DI water. Both filter devices then underwent post-use FIT at 22±4° C.

The acceptance criteria for a passing FIT is the rated bubble point for the capsule filter, which is provided by the manufacturer [≥4000 mbar (≥58 psi) air with water].

Results are provided at Table 3.

TABLE 3
Filter Integrity Testing Results for Capsule Filters

| | Manufacturer Rated | Pre-Use Bubble | Post-Use Bubble |
|---|---|---|---|
| Filter | Bubble Point^{a} | Point | Point^{b} |
| 1 | ≥58 psi air with water | >80 psi | >80 psi |
| 2 | ≥58 psi air with water | >80 psi | >80 psi |

^{a}Bubble Point at 23° C.
^{b}Post-use testing occurred after 48-hour exposure to PCV20 MDV conjugate pool

The results of this study demonstrate that there is no impact to the filter integrity of the polyethersulfone (PES) membrane (0.5 μm/0.2 μm) filter when exposed for at least 48 hours to the conjugate pool formulation. Both filters passed pre and post use integrity test.

The compatibility testing is also applicable to other filter sizes in the same filter family.

### Example 3 Rate of Addition and Mixing of 2-Phenoxyethanol in a Multivalent Conjugate Pool

The objective of this study was to determine the rate of addition and the speed of mixing required to dissolve 2-PE in a conjugate pool without significantly increasing the turbidity of the intermediate solution.

Formulations of a 20-valent conjugate pool (see example 2) with 17 mg/mL and 20 mg/mL 2-PE were shown to have a significant increase in turbidity. For both formulations, 2-PE was added to the conjugate pool without controlling the rate of 2-PE addition or the mixing of the conjugate pool during and after addition. It was hypothesized that these two factors play a role in the observed turbidity increase.

A study was performed to understand whether there is a need to control the 2-PE addition to the conjugate pool.

The study evaluated the potential role of the rate of 2-PE addition and mixing speed in the dissolution of 2-PE in the conjugate pool.

1.97 L of a 20-valent conjugate pool (see example 2) was formulated without the addition of 2-PE, with the final volume targeted for 2 L and final 2-PE concentration targeted for 15 mg/mL. The bulk conjugate pool was separated in 197 mL aliquots to add 2-PE to the individual aliquots with varying rates of addition and mixing.

The aliquots evaluated are listed in Table 4:

TABLE 4
Conjugate Pool aliquots

| | | | Total | | Scaling | | |
|---|---|---|---|---|---|---|---|
| | Rate of | | Addition | | Rate of | | |
| Aliquot | Addition | | Time | | Addition | | Mixing Parameter |
| 20-valent Conjugate | 0 | | 0 | | 0 | | N/A |
| Pool | | | | | | | |
| Instant 2-PE Addition | N/A | | ~3 | seconds | N/A | | 30 min delay, |
| with Delayed Mixing | | | | | | | continuous |
| | | | | | | | mixing at 200 RPM |
| | | | | | | | for 2 hours |
| Instant 2-PE Addition | N/A | | ~3 | seconds | N/A | | Continuous |
| with Instant Mixing | | | | | | | mixing at 200 |
| | | | | | | | RPM for 2 hours |
| 3 mL/min 2-PE | 3 | mL/min | 0.9 | min | 15.1 | mL/min/L | Continuous |
| Addition | | | | | | | mixing at 200 |
| with Instant Mixing | | | | | | | RPM for 2 hours |
| 1 mL/min 2-PE | 1 | mL/min | 2.71 | min | 5 | mL/min/L | Continuous |
| Addition | | | | | | | mixing at 200 |
| with Instant Mixing | | | | | | | RPM for 2 hours |
| 0.1 mL/min 2-PE | 0.1 | mL/min | 27.1 | min | 0.5 | mL/min/L | Continuous |
| Addition | | | | | | | mixing at 200 |
| with Instant Mixing | | | | | | | RPM for 2 hours |

For each aliquot, 197 mL of conjugate pool was added to a 250 mL glass beaker. Each beaker was placed on a mixing plate and a magnetic stir was added. The Instant Addition aliquots of 2.71 mL of 2-PE (3 g of 2-PE added, 2-PE density is 1.107 g/ml) were added instantaneously (i.e. all at once) to the conjugate pool manually using a 5 mL disposable syringe with a 21 G 1″ stainless steel needle. For the controlled rate of addition aliquots, 2-PE (same amount) was added into the mixing conjugate pool using the same syringe and needle but the rate of dispensing 2-PE was controlled using an automated syringe pump. The syringe pump was programed to add 2-PE from the syringe at the rates specified above. The mixing plate was set at 200 RPM for 2 hours for all aliquots. The Instant Addition with Delayed Mixing had the mixing delayed by 30 minutes after 2-PE addition was complete.

A qualitative approach was utilized by evaluating the visual appearance of the aliquoted conjugate pools after 2-PE dissolution. Videos were taken to capture the addition of 2-PE into the aliquoted conjugated pools. Pictures were taken after a 2 hour mix post-addition of 2-PE for all aliquots and appearance was compared to one another in an appearance box.

While the 1 mL/min Addition with Instant Mixing and 0.1 mL/min Addition with Instant Mixing aliquots appeared to eliminate the observed turbidity, slight increased turbidity compared to the control conjugate pool (without 2-PE) was observed in these aliquots when held near the light source.

The results of this study demonstrate that the degree of turbidity in the conjugate pool after addition of 2-PE is dependent on the rate of addition and time of mixing. The visual appearance of the Instant Addition with Delayed Mixing aliquot was significantly more turbid than the Instant Addition with Instant Mixing aliquot, suggesting that mixing while adding 2-PE to the conjugate pool had a direct impact on turbidity formation. The controlled rate of addition aliquots were significantly less turbid than the Instant Addition aliquots. Furthermore, slower rate of addition of 2-PE correlated with decreased turbidity in the conjugate pool after 2-PE dissolution was complete.

### Example 4 Evaluation of 2-Phenoxyethanol (2-PE) Addition to Formulate a Multivalent Conjugate Vaccine

The objective of this study was to evaluate addition of 2-PE in the process of formulating a 20-valent conjugate vaccine adjuvanted with alum. 2-PE has been added either to: 1-the final formulated bulk vaccine or 2-and 3-to the conjugate pool before final formulation at two different rates.

The process steps that were primarily evaluated were 2-PE addition, filtration, and addition of adjuvant (AIPO₄).

### 1:2-PE Added to the Final Formulated Bulk Vaccine

A 20-valent conjugate pool (see example 2) was mixed at 200 rpm and maintained for at least 5 minutes.

A pre-filtration sample was taken (135 ml). The conjugate pool sample was filtered (0.5/0.2 μm Polyethersulfone (PES) filter) using a product volume: filter surface area ratio (L/m2) of about 76.3. Filtration was initiated using a peristaltic pump. After approximately half of the total volume was filtered the filtration operation was paused by stopping the peristaltic pump. The product was held in the filter for at least 30 minutes before re-starting the filtration operation to evaluate a filtration pause during manufacturing.

A post-filtration sample was taken.

Succinate buffered saline and AIPO₄ were added (final concentration 5 mM succinate buffer (pH 5.8), 150 mM sodium chloride, and 0.25 mg/ml of aluminum as aluminum phosphate (AIPO4)), and the final drug product was mixed for at least 120 minutes.

2-PE was added using a syringe pump and the total time of the 2-PE addition was recorded—see Table 5 for 2-PE addition parameters.

After 120 minutes of mixing post-2-PE addition, the mixing was stopped.

2:2-PE was Added to the Conjugate Pool at Rate of 5.0 ml/min/L (5.5 g/min/L of 2-PE Added) Before Final Formulation

4 L of a 20-valent conjugate pool (see example 2) has been added in a 5 L glass vessel. Mixing at 200 rpm has been initiated and maintained for at least 5 minutes. 2-PE has been added using a syringe pump at 20 mL/min—see Table 5 for 2-PE addition parameters.

After 2-PE addition was complete, samples were taken from the top/bottom of the vessel.

The 2-PE dissolution time determined by visual observation was recorded—see Table 7.

After 120 minutes of mixing, the mixing was stopped.

A pre-filtration sample was taken (135 ml) and photographs were taken.

The conjugate pool sample was filtered (0.5/0.2 μm Polyethersulfone (PES) filter) using a product volume: filter surface area ratio (L/m2) of about 76.3. Filtration was initiated using a peristaltic pump. After approximately half of the total volume was filtered the filtration operation was paused by stopping the peristaltic pump. The product was held in the filter for at least 30 minutes before re-starting the filtration operation and the filter hold time was determined.

A post-filtration sample was taken.

Succinate buffered saline and AIPO₄ were added (final concentration 5 mM succinate buffer (pH 5.8), 150 mM sodium chloride, and 0.25 mg/ml of aluminum as aluminum phosphate (AIPO4)), and the final drug product was mixed for at least 120 minutes.

### 3: Fast 2-PE Addition to the Conjugate Pool Before Final Formulation

400 mL of a 20-valent conjugate pool (see example 2) has been added in a 500 mL glass vessel. Mixing at 200 rpm has been initiated and maintained for at least 5 minutes.

2-PE has been added using a syringe (added as quick as possible) and recorded total time and amount of the 2-PE addition—see Table 5 for 2-PE addition parameters.

After 120 minutes of mixing, the mixing was stopped.

A pre-filtration sample was taken (135 ml) and photographs were taken. The conjugate pool sample was filtered (0.5/0.2 μm Polyethersulfone (PES) filter) using a product volume: filter surface area ratio (L/m2) of about 76.3. Filtration was initiated using a peristaltic pump. After approximately half of the total volume was filtered the filtration operation was paused by stopping the peristaltic pump. The product was held in the filter for at least 30 minutes before re-starting the filtration operation and the filter hold time was determined.

A post-filtration sample was taken.

Succinate buffered saline and AIPO₄ were added (final concentration 5 mM succinate buffer (pH 5.8), 150 mM sodium chloride, and 0.25 mg/mL of aluminum as aluminum phosphate (AIPO4)), and the final Drug Product (DP) was mixed for at least 120 minutes.

TABLE 5
2-PE Addition parameters

| | 2-PE added | | 2-PE added | | Fast | |
|---|---|---|---|---|---|---|
| | to final | | at rate of | | 2-PE | |
| Parameter | bulk | | 5.0 mL/min/L | | addition | |
| 2-PE added (g) | 1.35 | | 60 | g | 6 | g |
| 2-PE addition | 2.7 | min | 2.7 | min | 0.13 | min |
| time | | | | | | |
| 2-PE density | 1.107 | g/mL | 1.107 | g/mL | 1.107 | g/mL |
| Conjugate pool | N/A | | 4 | L | 0.4 | L |
| volume after | | | | | | |
| 2-PE addition | | | | | | |
| Bulk DP | 0.15 | L | N/A | | N/A | |
| volume | | | | | | |
| after 2-PE | | | | | | |
| addition | | | | | | |
| 2-PE rate of | 3.0 | mL/min/L | 5.0 | mL/min/L | 104.2 | mL/min/L |

| addition | | | |
|---|---|---|---|
| Mixing speed | 200 | 200 | 200 |
| stirring (rpm) | | | |

### Results:

This study evaluated options of 2-PE addition by formulating batches, including 2-PE addition to conjugate pool, fast 2-PE addition to conjugate pool, and 2-PE addition to final drug product. For addition of 2-PE to the conjugate pool, the data demonstrated a uniform spread of 2-PE into the bulk material. No significant turbidity was observed in any of the three pre-filtration samples. Various quality attributes of the drug products were assessed, and drug products met all acceptance criteria. These results demonstrate the feasibility of 2-PE addition to the conjugate pool (before AIPO₄ addition and before sterile filtration).

### Aspects of the Invention

The following clauses describe additional embodiments of the invention:
C1. A process for the production of a conjugate vaccine comprising a preservative, said process comprising the steps of:
(a) adding a preservative from a bulk solution to a solution comprising one or more conjugate(s), wherein said preservative is added without sterile filtration; and
(b) subsequently sterile filter the mixture comprising said preservative and said conjugate(s).
C2. The process of C1, further comprising the step of (c) subsequently adding an adjuvant.
C3. The process of C2, wherein the adjuvant is aluminum phosphate.
C4. The process of C3, wherein the final concentration of aluminum phosphate in the vaccine is between about 0.1 mg/mL and about 0.5 mg/ml.
C5. The process of C4, wherein the final concentration of aluminum phosphate in the vaccine is about 0.25 mg/ml.
C6. The process of anyone of C1-C5, wherein said preservative is hydrophobic and viscous at the concentration of the bulk solution.
C7. The process of anyone of C1-C6, wherein the viscosity of the preservative at the concentration of the bulk solution is at least 10 centistokes at 25° C.
C8. The process of anyone of C1-C6, wherein the viscosity of the preservative at the concentration of the bulk solution is at least 15 centistokes at 25° C.
C9. The process of anyone of C1-C6, wherein the viscosity of the preservative at the concentration of the bulk solution is at least 20 centistokes at 25° C.
C10. The process of anyone of C1-C6, wherein the viscosity of the preservative at the concentration of the bulk solution is between about 10 to about 50 centistokes at 25° C.
C11. The process of anyone of C1-C6, wherein the viscosity of the preservative at the concentration of the bulk solution is between about 15 to about 25 centistokes at 25° C.
C12. The process of anyone of C1-C11, wherein the preservative is 2-phenoxyethanol (2-PE), phenol, meta-cresol, methyl-paraben, propyl-paraben or thiomersal.
C13. The process of anyone of C1-C11, wherein the preservative is 2-phenoxyethanol (2-PE) or thiomersal.
C14. The process of anyone of C1-C13, wherein the preservative is added undiluted.
C15. The process of anyone of C1-C14, wherein the bulk solution of preservative is the undiluted pure preservative.
C16. The process of anyone of C1-C15, wherein the preservative is 2-phenoxyethanol (2-PE).
C17. The process of anyone of C1-C16, wherein the bulk solution is neat 2-phenoxyethanol (2-PE).
C18. The process of C17, wherein the 2-PE is diluted by between about 10-fold and 200-fold in the solution following step (a).
C19. The process of C17, wherein the 2-PE is diluted by about 50-fold, about 60-fold, about 70-fold, or about 80-fold in the solution following step (a).
C20. The process of C17, wherein the 2-PE is diluted by between about 73-fold and 74-fold in the solution following step (a).
C21. The process of anyone of C16-C20, wherein the concentration of 2-PE in the solution following step (a) is between about 1 mg/ml and about 25 mg/ml.
C22. The process of anyone of C16-C20, wherein the concentration of 2-PE in the solution following step (a) is between about 10 mg/ml and about 20 mg/ml.
C23. The process of anyone of C16-C20, wherein the concentration of 2-PE in the solution following step (a) is between about 15 mg/ml and about 17 mg/ml.
C24. The process anyone of C16-C20, wherein the concentration of 2-PE in the solution following step (a) is about 15 mg/ml.
C25. The process of anyone of C16-C20, wherein the concentration of 2-PE in the solution following step (a) is about 17 mg/ml.
C26. The process of anyone of C16-C25, wherein 2-PE is added at a rate of addition of between about 0.5 ml/min per L of solution comprising one or more conjugate(s) to about 5.0 ml/min per L of solution comprising one or more conjugate(s).
C27. The process of anyone of C16-C25, wherein 2-PE is added at a rate of addition of between about 1 ml/min per L of solution comprising one or more conjugate(s) to about 4.0 ml/min per L of solution comprising one or more conjugate(s).
C28. The process of anyone of C17-C27, wherein neat 2-PE is added using a pump.
C29. The process of anyone of C17-C27, wherein neat 2-PE is added using a peristaltic pump.
C30. The process of anyone of C17-C27, wherein neat 2-PE is added using a peristaltic pump using silicone or polyurethane tubing.
C31. The process of anyone of C17-C27, wherein neat 2-PE is added using a peristaltic pump using thermoplastic elastomer tubing.
C32. The process of anyone of C16-C31, wherein the final concentration of 2-PE in the vaccine is between about 5 mg/ml to about 15 mg/ml.
C33. The process of anyone of C16-C31, wherein the final concentration of 2-PE in the vaccine is between about 7 mg/ml to about 12 mg/ml.
C34. The process of anyone of C16-C31, wherein the final concentration of 2-PE in the vaccine is about 10 mg/ml.
C35. The process of anyone of C16-C31, wherein the final concentration of 2-PE in the vaccine is about 9 mg/ml.
C36. The process of anyone of C1-C35, wherein the volume of the solution following step (a) is between about 100 L and about 500 L.
C37. The process of anyone of C1-C35, wherein the volume of the solution following step (a) is about 180 L.
C38. The process of anyone of C1-C37, wherein the preservative is added under continuous mixing.
C39. The process of C38, wherein there is no delay between the addition of the preservative and the start of mixing.
C40. The process of anyone of C1-C39, wherein the preservative is added under continuous mixing where the angular velocity is between about 50 rpm to about 500 rpm.
C41. The process of anyone of C1-C39, wherein the preservative is added under continuous mixing where the angular velocity is between about 100 rpm to about 400 rpm.
C42. The process of anyone of C1-C39, wherein the preservative is added under continuous mixing where the angular velocity is between about 150 rpm to about 300 rpm.
C43. The process of anyone of C1-C39, wherein the preservative is added under continuous mixing where the angular velocity is between about 150 rpm to about 200 rpm.
C44. The process of anyone of C1-C39, wherein the preservative is added under continuous mixing where the angular velocity is about 150 rpm.
C45. The process of anyone of C1-C39, wherein the preservative is added under continuous mixing where the angular velocity is about 200 rpm.
C46. The process of anyone of C1-C45, wherein following the addition of preservative and before sterile filtration, the solution is mixed for between about 15 minutes to about 5 hours.
C47. The process of anyone of C1-C45, wherein following the addition of preservative and before sterile filtration, the solution is mixed for between about 30 minutes to about 3 hours.
C48. The process of anyone of C1-C45, wherein following the addition of preservative and before sterile filtration, the solution is mixed for between about 1 hour to about 3 hours.
C49. The process of anyone of C1-C45, wherein following the addition of preservative 15 and before sterile filtration, the solution is mixed about 2 hours.
C50. The process of anyone of C1-C49, wherein following the addition of preservative and before sterile filtration, the solution is mixed at an angular velocity between about 50 rpm and about 500 rpm.
C51. The process of anyone of C1-C49, wherein following the addition of preservative and before sterile filtration, the solution is mixed at an angular velocity between about 100 rpm and about 400 rpm.
C52. The process of anyone of C1-C49, wherein following the addition of preservative and before sterile filtration, the solution is mixed at an angular velocity between about 150 rpm and about 300 rpm.
C53. The process of anyone of C1-C52, wherein the solution to which the 30 preservative is added is a mono-valent composition.
C54. The process of anyone of C1-C52, wherein the solution to which the preservative is added is a bi-valent, tri-valent, tetra-valent, pent-valent or hexa-valent composition.
C55. The process of anyone of C1-C52, wherein the solution to which the preservative is added is a multi-valent composition comprising seven conjugates or more.
C56. The process of anyone of C1-C52, wherein the solution to which the preservative is added is a 7 to 25 valent composition.
C57. The process of anyone of C1-C52, wherein the solution to which the preservative is added is a 13 to 25 valent composition.
C58. The process of anyone of C1-C52, wherein the solution to which the preservative is added is a 15-valent composition.
C59. The process of anyone of C1-C52, wherein the solution to which the preservative is added is a 16-valent composition.
C60. The process of anyone of C1-C52, wherein the solution to which the preservative is added is a 20-valent composition.
C61. The process of anyone of C1-C52, wherein the solution to which the preservative is added is a 21-valent composition.
C62. The process of anyone of C1-C52, wherein the solution to which the preservative is added is a multi-valent pneumococcal conjugates composition.
C63. The process of anyone of C1-C52, wherein the solution to which the preservative is added is a pneumococcal conjugate composition comprising from 7 to 25 glycoconjugates from different serotypes of S. pneumoniae.
C64. The process of anyone of C1-C52, wherein the solution to which the preservative is added is a pneumococcal conjugate composition comprising conjugates from 15, 16, 17, 18, 19, 20, 21, 22, 23, 24 or 25 different serotypes of S. pneumoniae.
C65. The process of anyone of C1-C52, wherein the solution to which the preservative is added is a pneumococcal conjugate composition comprising conjugates from 20 different serotypes of S. pneumoniae.
C66. The process of anyone of C1-C52, wherein the solution to which the preservative is added is a 7, 8, 9, 10, 11, 12, 13, 14, 15, 16, 17, 18, 19 or 20-valent pneumococcal conjugates composition.
C67. The process of anyone of C1-C52, wherein the solution to which the preservative is added is a 15-valent pneumococcal conjugates composition.
C68. The process of anyone of C1-C52, wherein the solution to which the preservative is added is a 20-valent pneumococcal conjugates composition.
C69. The process of anyone of C1-C52, wherein the solution to which the preservative is added is a 21-valent pneumococcal conjugates composition.
C70. The process of anyone of C1-C52, wherein the solution to which the preservative is added is a 22-valent pneumococcal conjugates composition.
C71. The process of anyone of C1-C52, wherein the solution to which the preservative is added is a 21, 22, 23, 24 or 25-valent pneumococcal conjugates composition.
C72. The process of anyone of C1-C52, wherein the solution to which the preservative is added comprises glycoconjugates from S. pneumoniae serotypes 4, 6B, 9V, 14, 18C, 19F and 23F.
C73. The process of anyone of C1-C52, wherein the solution to which the preservative is added comprises glycoconjugates from S. pneumoniae serotypes 1, 4, 5, 6B, 7F, 9V, 14, 18C, 19F and 23F.
C74. The process of anyone of C1-C52, wherein the solution to which the preservative is added comprises glycoconjugates from S. pneumoniae serotypes 1, 3, 4, 5, 6B, 7F, 9V, 14, 18C, 19F and 23F.
C75. The process of anyone of C1-C52, wherein the solution to which the preservative is added comprises glycoconjugates from S. pneumoniae serotypes 1, 3, 4, 5, 6A, 6B, 7F, 9V, 14, 18C, 19A, 19F and 23F.
C76. The process of anyone of C1-C52, wherein the solution to which the preservative is added comprises glycoconjugates from S. pneumoniae serotypes 1, 3, 4, 5, 6A, 6B, 7F, 9V, 14, 18C, 19A, 19F, 22F, 23F and 33F.
C77. The process of anyone of C1-C52, wherein the solution to which the preservative is added comprises glycoconjugates from S. pneumoniae serotypes 1, 3, 4, 5, 6A, 6B, 7F, 8, 9V, 10A, 11A, 12F, 14, 15B, 18C, 19A, 19F, 22F, 23F and 33F.
C78. The process of anyone of C1-C52, wherein the solution to which the preservative is added comprises glycoconjugates from S. pneumoniae serotypes 1, 2, 3, 4, 5, 6A, 6B, 7F, 8, 9V, 10A, 11A, 12F, 14, 15B, 18C, 19A, 19F, 22F, 23F and 33F.
C79. The process of anyone of C1-C52, wherein the solution to which the preservative is added comprises glycoconjugates from S. pneumoniae serotypes 1, 3, 4, 5, 6A, 6B, 7F, 8, 9N, 9V, 10A, 11A, 12F, 14, 15B, 18C, 19A, 19F, 22F, 23F and 33F.
C80. The process of anyone of C1-C52, wherein the solution to which the preservative is added is a 10-valent pneumococcal glycoconjugate composition wherein said 10 glycoconjugates are from S. pneumoniae serotypes 1, 4, 5, 6B, 7F, 9V, 14, 18C, 19F and 23F.
C81. The process of anyone of C1-C52, wherein the solution to which the preservative is added is a 13-valent pneumococcal glycoconjugate composition wherein said 13 glycoconjugates are from S. pneumoniae serotypes 1, 3, 4, 5, 6A, 6B, 7F, 9V, 14, 18C, 19A, 19F and 23F.
C82. The process of anyone of C1-C52, wherein the solution to which the preservative is added is a 15-valent pneumococcal glycoconjugate composition wherein said 15 glycoconjugates are from S. pneumoniae serotypes 1, 3, 4, 5, 6A, 6B, 7F, 9V, 14, 18C, 19A, 19F, 22F, 23F and 33F.
C83. The process of anyone of C1-C52, wherein the solution to which the preservative is added is a 20-valent pneumococcal glycoconjugate composition wherein said 20 glycoconjugates are from S. pneumoniae serotypes 1, 3, 4, 5, 6A, 6B, 7F, 8, 9V, 10A, 11A, 12F, 14, 15B, 18C, 19A, 19F, 22F, 23F and 33F.
C84. The process of anyone of C1-C52, wherein the solution to which the preservative is added is a 21-valent pneumococcal glycoconjugate composition wherein said 21 glycoconjugates are from S. pneumoniae serotypes 1, 2, 3, 4, 5, 6A, 6B, 7F, 8, 9V, 10A, 11A, 12F, 14, 15B, 18C, 19A, 19F, 22F, 23F and 33F.
C85. The process of anyone of C1-C52, wherein the solution to which the preservative is added is a 21-valent pneumococcal glycoconjugate composition wherein said 21 glycoconjugates are from S. pneumoniae serotypes 1, 3, 4, 5, 6A, 6B, 7F, 8, 9N, 9V, 10A, 11A, 12F, 14, 15B, 18C, 19A, 19F, 22F, 23F and 33F.
C86. The process of anyone of C1-C52, wherein the solution to which the preservative is added is a 22-valent pneumococcal glycoconjugate composition wherein said 22 glycoconjugates are from S. pneumoniae serotypes 1, 2, 3, 4, 5, 6A, 6B, 7F, 8, 9N, 9V, 10A, 11A, 12F, 14, 15B, 18C, 19A, 19F, 22F, 23F and 33F.
C87. The process of anyone of C1-C52, wherein the solution to which the preservative is added is a 21-valent pneumococcal glycoconjugate composition wherein said 21 glycoconjugates are from S. pneumoniae serotypes 1, 3, 4, 5, 6A, 6B, 7F, 8, 9V, 10A, 11A, 12F, 14, 15A, 15B, 18C, 19A, 19F, 22F, 23F and 33F.
C88. The process of anyone of C1-C52, wherein the solution to which the preservative is added is a 21-valent pneumococcal glycoconjugate composition wherein said 21 glycoconjugates are from S. pneumoniae serotypes 1, 3, 4, 5, 6A, 6B, 7F, 8, 9V, 10A, 11A, 12F, 14, 15B, 18C, 19A, 19F, 22F, 23A, 23F and 33F.
C89. The process of anyone of C1-C52, wherein the solution to which the preservative is added is a 21-valent pneumococcal glycoconjugate composition wherein said 21 glycoconjugates are from S. pneumoniae serotypes 1, 3, 4, 5, 6A, 6B, 7F, 8, 9V, 10A, 11A, 12F, 14, 15B, 18C, 19A, 19F, 22F, 23B, 23F and 33F.
C90. The process of anyone of C1-C52, wherein the solution to which the preservative is added is a 21-valent pneumococcal glycoconjugate composition wherein said 21 glycoconjugates are from S. pneumoniae serotypes 1, 3, 4, 5, 6A, 6B, 7F, 8, 9V, 10A, 11A, 12F, 14, 15B, 18C, 19A, 19F, 22F, 23F, 24F and 33F.
C91. The process of anyone of C1-C52, wherein the solution to which the preservative is added is a 21-valent pneumococcal glycoconjugate composition wherein said 21 glycoconjugates are from S. pneumoniae serotypes 1, 3, 4, 5, 6A, 6B, 7F, 8, 9V, 10A, 11A, 12F, 14, 15B, 18C, 19A, 19F, 22F, 23F, 33F and 35B.
C92. The process of anyone of C1-C52, wherein the solution to which the Preservative is added is a 20-valent pneumococcal glycoconjugate composition wherein said 20 glycoconjugates are from S. pneumoniae serotypes 1, 3, 4, 5, 6A, 6B, 7F, 8, 9V, 10A, 11A, 12F, 14, 15B, 18C, 19A, 19F, 22F, 23F and 33F.
C93. The process of anyone of C63-C92, wherein the carrier protein of the glycoconjugate is TT, CRM197 or a C5a peptidase from Streptococcus (SCP).
C94. The process of anyone of C63-C92, wherein the carrier protein of the glycoconjugate is CRM₁₉₇.
C95. The process of anyone of C63-C92, wherein at least one capsular polysaccharide is conjugated to TT and the other capsular polysaccharides are all conjugated to CRM₁₉₇.
C96. The process of anyone of C63-C92, wherein one capsular polysaccharide is conjugated to TT and the other capsular polysaccharides are all conjugated to CRM₁₉₇.
C97. The process of anyone of C63-C92, wherein at least two capsular polysaccharides are conjugated to TT and the other capsular polysaccharides are all conjugated to CRM₁₉₇.
C98. The process of anyone of C63-C92, wherein two capsular polysaccharides are conjugated to TT and the other capsular polysaccharides are all conjugated to CRM₁₉₇.
C99. The process of anyone of C63-C92, wherein at least three capsular polysaccharides are conjugated to TT and the other capsular polysaccharides are all conjugated to CRM₁₉₇.
C100. The process of anyone of C63-C92, wherein three capsular polysaccharides are conjugated to TT and the other capsular polysaccharides are all conjugated to CRM₁₉₇.
C101. The process of anyone of C63-C92, wherein four capsular polysaccharides are conjugated to TT and the other capsular polysaccharides are all conjugated to CRM₁₉₇.
C102. The process of anyone of C1-C52, wherein the solution to which the preservative is added is a 21-valent pneumococcal conjugate composition wherein said 21 glycoconjugates are from S. pneumoniae serotypes 1, 3, 4, 5, 6A, 6B, 7F, 8, 9N, 9V, 10A, 11A, 12F, 14, 15B, 18C, 19A, 19F, 22F, 23F and 33F, wherein four capsular polysaccharides are conjugated to TT wherein the four capsular polysaccharides that are conjugated to TT are serotypes 15B, 22F and the other two serotypes are selected from the group consisting of serotypes 1, 3 and 5 and the other glycoconjugates are all conjugated to CRM₁₉₇.
C103. The process of anyone of C1-C52, wherein the solution to which the preservative is added is a 21-valent pneumococcal conjugate composition wherein said 21 glycoconjugates are from S. pneumoniae serotypes 1, 3, 4, 5, 6A, 6B, 7F, 8, 9N, 9V, 10A, 11A, 12F, 14, 15B, 18C, 19A, 19F, 22F, 23F and 33F, wherein four capsular polysaccharides are conjugated to TT wherein the four capsular polysaccharides that are conjugated to TT are serotypes 1, 5, 15B and 22F and the other glycoconjugates are all conjugated to CRM₁₉₇.
C104. The process of anyone of C1-C52, wherein the solution to which the preservative is added is a 21-valent pneumococcal conjugate composition wherein said 21 glycoconjugates are from S. pneumoniae serotypes 1, 3, 4, 5, 6A, 6B, 7F, 8, 9N, 9V, 10A, 11A, 12F, 14, 15B, 18C, 19A, 19F, 22F, 23F and 33F, wherein four capsular polysaccharides are conjugated to TT wherein the four capsular polysaccharides that are conjugated to TT are serotypes 1, 3, 15B and 22F and the other glycoconjugates are all conjugated to CRM₁₉₇.
C105. The process of anyone of C1-C52, wherein the solution to which the preservative is added is a 21-valent pneumococcal conjugate composition wherein said 21 glycoconjugates are from S. pneumoniae serotypes 1, 3, 4, 5, 6A, 6B, 7F, 8, 9N, 9V, 10A, 11A, 12F, 14, 15B, 18C, 19A, 19F, 22F, 23F and 33F, wherein four capsular polysaccharides are conjugated to TT wherein the four capsular polysaccharides that are conjugated to TT are serotypes 3, 5, 15B and 22F and the other glycoconjugates are all conjugated to CRM₁₉₇.
C106. The process of anyone of C1-C52, wherein the solution to which the preservative is added is a 21-valent pneumococcal conjugate composition wherein said 21 glycoconjugates are from S. pneumoniae serotypes 1, 3, 4, 5, 6A, 6B, 7F, 8, 9N, 9V, 10A, 11A, 12F, 14, 15B, 18C, 19A, 19F, 22F, 23F and 33F, wherein two glycoconjugates selected from serotypes 1, 3 and 5 are conjugated to TT and the other glycoconjugates are all conjugated to CRM₁₉₇.
C107. The process of anyone of C1-C52, wherein the solution to which the preservative is added is a 21-valent pneumococcal conjugate composition wherein said 21 glycoconjugates are from S. pneumoniae serotypes 1, 3, 4, 5, 6A, 6B, 7F, 8, 9N, 9V, 10A, 11A, 12F, 14, 15B, 18C, 19A, 19F, 22F, 23F and 33F, wherein glycoconjugates from serotypes 1 and 3 are conjugated to TT and the other glycoconjugates are all conjugated to CRM₁₉₇.
C108. The process of anyone of C1-C52, wherein the solution to which the preservative is added is a 21-valent pneumococcal conjugate composition wherein said 21 glycoconjugates are from S. pneumoniae serotypes 1, 3, 4, 5, 6A, 6B, 7F, 8, 9N, 9V, 10A, 11A, 12F, 14, 15B, 18C, 19A, 19F, 22F, 23F and 33F, wherein glycoconjugates from serotypes 1 and 5 are conjugated to TT and the other glycoconjugates are all conjugated to CRM₁₉₇.
C109. The process of anyone of C1-C52, wherein the solution to which the preservative is added is a 21-valent pneumococcal conjugate composition wherein said 21 glycoconjugates are from S. pneumoniae serotypes 1, 3, 4, 5, 6A, 6B, 7F, 8, 9N, 9V, 10A, 11A, 12F, 14, 15B, 18C, 19A, 19F, 22F, 23F and 33F, wherein glycoconjugates from serotypes 3 and 5 are conjugated to TT and the other glycoconjugates are all conjugated to CRM₁₉₇.
C110. The process of anyone of C1-C52, wherein the solution to which the preservative is added is a 15-valent pneumococcal conjugate composition wherein said 15 glycoconjugates are from S. pneumoniae serotypes 1, 3, 4, 5, 6A, 6B, 7F, 9V, 14, 18C, 19A, 19F, 22F, 23F and 33F, wherein all the glycoconjugates are conjugated to CRM₁₉₇.
C111. The process of anyone of C1-C52, wherein the solution to which the preservative is added is a 20-valent pneumococcal conjugate composition wherein said 20 glycoconjugates are from S. pneumoniae serotypes 1, 3, 4, 5, 6A, 6B, 7F, 8, 9V, 10A, 11A, 12F, 14, 15B, 18C, 19A, 19F, 22F, 23F and 33F, wherein all the glycoconjugates are conjugated to CRM₁₉₇.
C112. The process of anyone of C1-C111, wherein the solution to which the preservative is added comprises a pharmaceutically acceptable excipient, carrier, or diluent.
C113. The process of anyone of C1-C111, wherein the solution to which the preservative is added comprise one or more of a buffer, a salt, a divalent cation, a non-ionic detergent, a cryoprotectant such as a sugar, and an antioxidant such as a free radical scavenger or chelating agent, or any multiple combinations thereof.
C114. The process of anyone of C1-C111, wherein the solution to which the preservative is added comprises a buffer.
C115. The process of C114, wherein said buffer has a pKa of about 3.5 to about 7.5.
C116. The process of C114, wherein said buffer is phosphate, succinate, histidine or citrate.
C117. The process of C114, wherein said buffer is succinate.
C118. The process of C114, wherein said buffer is histidine.
C119. The process of C114, wherein said buffer is histidine at a concentration of 10 mM to 30 mM.
C120. The process of C114, wherein said buffer is histidine at a concentration of 15 mM to 25 mM.
C121. The process of C114, wherein said buffer is histidine at a concentration of about 22 mM.
C122. The process of C114, wherein said buffer is succinate at a concentration of 1 mM to 10 mM.
C123. The process of C114, wherein said buffer is succinate at a concentration of about 5.5 mM.
C124. The process of anyone of C1-C123, wherein the solution to which the preservative is added comprises a salt.
C125. The process of C124, wherein said salt is magnesium chloride, potassium chloride, sodium chloride or a combination thereof.
C126. The process of C124, wherein said salt is sodium chloride.
C127. The process of anyone of C1-C123, wherein the solution to which the preservative is added comprises sodium chloride at about 150 mM.
C128. The process of anyone of C1-C127, wherein the solution to which the preservative is added comprises a surfactant.
C129. The process of C128, wherein said surfactant is polysorbate 20, polysorbate 40, polysorbate 60, polysorbate 65, polysorbate 80, polysorbate 85, TRITON™ N-101, TRITON™ X-100, oxtoxynol 40, nonoxynol-9, triethanolamine, triethanolamine polypeptide oleate, polyoxyethylene-660 hydroxystearate (PEG-15, Solutol H 15), polyoxyethylene-35-ricinoleate (CREMOPHOR® EL), soy lecithin or a poloxamer.
C130. The process of C129, wherein said surfactant is polysorbate 80 or polysorbate 20.
C131. The process of C129, wherein said surfactant is polysorbate 20.
C132. The process of C131, wherein the concentration of polysorbate 20 in the solution is between 0.01% to 1% polysorbate 20 weight to weight (w/w).
C133. The process of C131, wherein the concentration of polysorbate 20 in the solution is between 0.1% to 0.5% polysorbate 20 weight to weight (w/w).
C134. The process of C131, wherein the concentration of polysorbate 20 in the solution is about 0.1%, about 0.2%, about 0.3%, about 0.4%, about 0.5%, or about 0.6% polysorbate 20 (w/w).
C135. The process of C131, wherein the concentration of the polysorbate 20 in the solution is about 0.33% polysorbate 20 (w/w).
C136. The process of C129, wherein said surfactant is polysorbate 80.
C137. The process of C136, wherein the concentration of polysorbate 80 in the solution is between 0.001% to 1% polysorbate 80 weight to weight (w/w).
C138. The process of C136, wherein the concentration of polysorbate 80 in the solution is between 0.01% to 0.5% polysorbate 80 weight to weight (w/w).
C139. The process of C136, wherein the concentration of polysorbate 80 in the solution is about 0.01%, about 0.02%, about 0.03%, about 0.04%, about 0.05%, or about 0.06% polysorbate 80 (w/w).
C140. The process of C136, wherein the concentration of polysorbate 80 in the solution is about 0.033% polysorbate 80 (w/w).
C141. The process of C136, wherein the final concentration of polysorbate 80 in the vaccine is between about 0.01% and about 0.03%.
C142. The process of C136, wherein the final concentration of polysorbate 80 in the vaccine is about 0.02%.
C143. The process of anyone of claims C1-C142, wherein the solution to which the preservative is added has a pH of between 5.5 to 7.5.
C144. The process of anyone of C1-C142, wherein the solution to which the preservative is added has a pH of between 5.6 to 7.0.
C145. The process of anyone of C1-C142, wherein the solution to which the preservative is added has a pH of between 5.8 to 6.0.
C146. The process of anyone of C1-C142, wherein the solution to which the preservative is added has a pH of about 5.8.
C147. The process of anyone of C1-C142, wherein the final pH in the vaccine is between about 5.3 and about 6.3.
C148. The process of anyone of C1-C142, wherein the final pH in the vaccine is about 5.8
C149. The process of anyone of C1-C52, wherein the solution to which the preservative is added is a 20-valent pneumococcal conjugate composition wherein said 20 glycoconjugates are from S. pneumoniae serotypes 1, 3, 4, 5, 6A, 6B, 7F, 8, 9V, 10A, 11A, 12F, 14, 15B, 18C, 19A, 19F, 22F, 23F and 33F, wherein the glycoconjugates are all conjugated to CRM197, which comprises between about 5 μg/ml to about 10 μg/ml of each of capsular polysaccharides serotypes 1, 3, 4, 5, 6A, 7F, 8, 9V, 10A, 11A, 12F, 14, 15B, 18C, 19A, 19F, 22F, 23F, and 33F and between about 10 μg/ml to about 20 μg/ml of the capsular polysaccharide of serotype 6B, between about 0.01% to about 0.05% (w/w) polysorbate 80, about 150 mM sodium chloride and between about 1 mM to about 10 mM succinate buffer pH 5.8.
C150. The process of anyone of C1-C52, wherein the solution to which the preservative is added is a 15-valent pneumococcal conjugate composition wherein said 15 glycoconjugates are from S. pneumoniae serotypes 1, 3, 4, 5, 6A, 6B, 7F, 9V, 14, 18C, 19A, 19F, 22F, 23F and 33F, wherein the glycoconjugates are all conjugated to CRM₁₉₇, which comprises between about 5 μg/ml to about 10 μg/ml of each of capsular polysaccharides serotypes 1, 3, 4, 5, 6A, 7F, 9V, 14, 18C, 19A, 19F, 22F, 23F, and 33F and between about 10 μg/ml to about 20 μg/ml of the capsular polysaccharide of serotype 6B, about 150 mM sodium chloride, between about 0.1% to about 0.5% (w/w) polysorbate 20 and between about 15 mM to about 25 mM L-Histidine buffer pH 5.8.
C151. The process of anyone of C1-C52, wherein the solution to which the preservative is added is a 20-valent pneumococcal conjugate composition wherein said 20 glycoconjugates are from S. pneumoniae serotypes 1, 3, 4, 5, 6A, 6B, 7F, 8, 9V, 10A, 11A, 12F, 14, 15B, 18C, 19A, 19F, 22F, 23F and 33F, wherein the glycoconjugates are all conjugated to CRM197, which comprises about 7.5 μg/ml of each of capsular polysaccharides serotypes 1, 3, 4, 5, 6A, 7F, 8, 9V, 10A, 11A, 12F, 14, 15B, 18C, 19A, 19F, 22F, 23F, and 33F and about 7.5 μg/ml of the capsular polysaccharide of serotype 6B, about 0.033% (w/w) polysorbate 80, about 150 mM sodium chloride and about 5.5 mM succinate buffer pH 5.8.
C152. The process of anyone of C1-C151, wherein the final protein concentration in the vaccine is between about 60 g/ml and about 130 μg/ml.
C153. The process of anyone of C1-C151, wherein the final protein concentration in the vaccine is between about 90 μg/ml and about 99 μg/ml.
C154. The process of anyone of C1-C153, wherein the final D10 particle size distribution of the vaccine is between about 4,000 and about 5,000.
C155. The process of anyone of C1-C153, wherein the final D10 particle size distribution of the vaccine is between about 4,300 and about 4,700.
C156. The process of anyone of C1-C153, wherein the final D50 particle size distribution of the vaccine is between about 7,000 and about 8,000.
C157. The process of anyone of C1-C153, wherein the final D50 particle size distribution of the vaccine is between about 7,500 and about 7,900.
C158. The process of anyone of C1-C153, wherein the final D90 particle size distribution of the vaccine is between about 12,000 and about 15,000.
C159. The process of anyone of C1-C153, wherein the final D90 particle size distribution of the vaccine is between about 12,300 and about 14,900.
C160. The process of anyone of C1-C159, wherein following step (a), the mixture comprising the preservative and the conjugate(s) is sterilely filtered.
C161. The process of C160, wherein the filter has a nominal retention range of between about 0.05-0.2 μm.
C162. The process of C160, wherein the filter has a nominal retention range of between about 0.1-0.2 μm.
C163. The process of C160, wherein the filter has a nominal retention range of between about 0.15-0.2 μm.
C164. The process of C160, wherein the filter has a nominal retention range of about 0.1, about 0.15 or about 0.2μm.
C165. The process of C160, wherein the filter has a nominal retention range of about 0.2μm.
C166. The process of anyone of C160-C165, wherein the filter comprises a prefilter.
C167. The process of C166, wherein the prefilter has a nominal retention range of between about 0.1 μm and 1 μm.
C168. The process of C166, wherein the prefilter has a nominal retention range of about 0.5 μm.
C169. The process of C166, wherein the prefilter has a nominal retention range of about 0.5 μm and the filter has a nominal retention range of about 0.2 μm.
C170. The process of anyone of C160-C169, wherein the filter has a filter capacity of about 25-1000 L/m2, 50-1000 L/m2, 75-1000 L/m2, 100-1000 L/m2, 150-1000 L/m2, 200-1000 L/m2, 250-1000 L/m2, 300-1000 L/m2, 350-1000 L/m2, 400-1000 L/m2, 500-1000 L/m2 or 750-1000 L/m2.
C171. The process of anyone of C160-C169, wherein, the filter has a filter capacity of 200-1000 L/m2, 250-1000 L/m2 or 300-1000 L/m2.
C172. The process of anyone of C160-C169, wherein, the filter has a filter capacity of about 300-1000 L/m2.
C173. The process of anyone of C160-C172, wherein the filter comprises polysulfone.
C174. The process of anyone of C160-C172, wherein the filter comprises polyethersulfone (PES).
C175. The process of anyone of C160-C174, wherein the filter is a capsule filter.
C176. The process of anyone of C1-C175, wherein, the conjugate vaccine obtained by the process comprises one or more conjugate and a preservative.

1. A process for the production of a conjugate vaccine comprising a preservative, said process comprising the steps of:
(a) adding a preservative from a bulk solution to a solution comprising one or more conjugate(s), a buffer, a salt, and a surfactant; and
(b) subsequently sterile filter the mixture comprising said preservative, conjugate(s), buffer, salt, and surfactant,
wherein said preservative is not sterile filtered before addition to said solution, and
wherein said preservative is hydrophobic and viscous at the concentration of the bulk solution.

2. The process of claim 1, further comprising the step of (c) subsequently adding an adjuvant.

3. The process of claim 2, wherein the adjuvant is aluminum phosphate.

4. The process of claim 3, wherein the final concentration of aluminum phosphate in the vaccine is about 0.25 mg/ml.

5. The process of anyone of claims 1-4, wherein the viscosity of the preservative at the concentration of the bulk solution is between about 15 and about 25 centistokes at 25° C.

6. The process of anyone of claims 1-5, wherein the preservative is 2-phenoxyethanol (2-PE), phenol, meta-cresol, methyl-paraben, propyl-paraben, or thiomersal.

7. The process of anyone of claims 1-6, wherein the bulk solution is neat 2-phenoxyethanol (2-PE).

8. The process of anyone of claims 1-7, wherein the preservative is 2-PE and the concentration of 2-PE in the solution following step (a) is between about 15 mg/ml and about 17 mg/ml.

9. The process of anyone of claims 1-8, wherein the final concentration of 2-PE in the vaccine is about 9 mg/ml or about 10 mg/ml.

10. The process of anyone of claims 1-9, wherein the buffer comprises succinate, the salt comprises sodium chloride, and the surfactant comprises polysorbate 80.

11. The process of claim 10, wherein:
(a) the concentration of succinate is between about 1 mM and about 10 mM;
(b) the concentration of sodium chloride is between about 100 mM and about 200 mM; and
(c) the concentration of polysorbate 80 is between about 0.01% and about 0.05%.

12. The process of anyone of claims 1-11, wherein the solution to which the preservative is added has a pH of between about 5.8 and about 6.0.

13. The process of anyone of claims 1-12, wherein the preservative is added at a rate of addition of between about 0.5 ml/min per L and about 5.0 ml/min per L of solution comprising one or more conjugate(s), buffer, salt, and surfactant.

14. The process of anyone of claims 1-13, wherein the preservative is added using a peristaltic pump.

15. The process of anyone of claims 1-14, wherein the preservative is added under continuous mixing and there is no delay between the addition of the preservative and the start of mixing, and wherein:
(a) the angular velocity of mixing is between about 150 rpm and about 200 rpm; or
(b) following the addition of preservative and before sterile filtration, the solution is mixed for between about 30 minutes and about 3 hours.

16. The process of anyone of claims 1-15, wherein the filtration of step (b) comprises use of a filter that has a nominal retention range of about 0.1 μm, about 0.15 μm, or about 0.2 μm.

17. The process of claim 16, wherein the filter comprises a prefilter with a nominal retention range of about 0.5 μm.

18. The process of anyone of claims 1-17, wherein the filtration of step (b) comprises use of a filter that has a filter capacity of 200-1000 L/m², 250-1000 L/m², or 300-1000 L/m².

19. The process of anyone of claims 1-18, wherein the filtration of step (b) comprises use of a filter comprising polyethersulfone (PES).

20. The process of anyone of claims 1-19, wherein the solution to which the preservative is added is a pneumococcal conjugate composition comprising from 7 to 25 glycoconjugates from different serotypes of S. pneumoniae.

21. The process of anyone of claims 1-19, wherein the solution to which the preservative is added is a 20-valent pneumococcal glycoconjugate composition wherein said 20 glycoconjugates are from S. pneumoniae serotypes 1, 3, 4, 5, 6A, 6B, 7F, 8, 9V, 10A, 11A, 12F, 14, 15B, 18C, 19A, 19F, 22F, 23F and 33F.

22. The process of claim 20 or 21, wherein all the glycoconjugates are conjugated to CRM₁₉₇.

23. The process of anyone of claims 1-19, wherein the solution to which the preservative is added is a 20-valent pneumococcal conjugate composition wherein said 20 glycoconjugates are from S. pneumoniae serotypes 1, 3, 4, 5, 6A, 6B, 7F, 8, 9V, 10A, 11A, 12F, 14, 15B, 18C, 19A, 19F, 22F, 23F and 33F, wherein the glycoconjugates are all conjugated to CRM₁₉₇, which comprises about 7.5 μg/ml of each of capsular polysaccharides serotypes 1, 3, 4, 5, 6A, 7F, 8, 9V, 10A, 11A, 12F, 14, 15B, 18C, 19A, 19F, 22F, 23F, and 33F and about 7.5 μg/ml of the capsular polysaccharide of serotype 6B, about 0.033% (w/w) polysorbate 80, about 150 mM sodium chloride and about 5.5 mM succinate buffer pH 5.8.
